(19) United States
(12) Patent Application Publication
(10) Pub. No.: US 2008/0124362 A1
(43) Pub. Date: May 29, 2008

(54) Domain II Mutants Of Anthrax Lethal Factor

(75) Inventor: Nicholas S. Duesbery, Grand Rapids, MI (US)

Correspondence Address: PRICE HENEVELD COOPER DEWITT & LITTON, LLP, 695 KENMOOR, S.E., P O BOX 2567, GRAND RAPIDS, MI, 49501, US

(73) Assignee: VAN ANDEL RESEARCH INSTITUTE, Grand Rapids, MI (US)

(21) Appl. No.: 11/576,112

(22) PCT Filed: Oct. 3, 2005
(86) PCT No.: PCT/US05/35722

§ 371 Date: Nov. 19, 2007

Related U.S. Application Data
(60) Provisional application No. 60/614,555, filed on Oct. 1, 2004.

(51) Int. Cl. C07K 14/32; C12N 15/31; G01N 33/53; A61K 39/07

(52) U.S. Cl. 424/236.1; 530/350; 536/23.7; 435/7.8; 424/246.1

(57) ABSTRACT

A series of mutants of Anthrax lethal factor (LF) are disclosed which define a conformational epitope or region of the molecule that interacts with the LF target, the MEK enzyme. Such mutants or variants, and nucleic acids encoding them are disclosed. The knowledge of such binding, separate from recognition of MEK by the protease active site of LF, serves as the basis for novel screening assays for discovery of inhibitors of this additional form of LF-MEK binding which is necessary for ultimate proteolysis and toxicity. The nontoxic LF mutants are useful as immunogenic compositions for generating antibodies and a state of immunity specific for the LF component of a B. arithracis infection or exposure otherwise to the anthrax lethal toxin.

### BACKGROUND OF THE INVENTION

1. Field of the Invention

The present invention in the fields of biochemistry, genetics and medicine is directed to mutants of anthrax Lethal Factor (LF) in domain II of the molecule that lack toxicity and are therefore useful in screening methods and as an immunogenic compositions against anthrax.

2. Description of the Background Art

Anthrax toxin is derived from an exotoxin produced by the gram-positive bacterium Bacillus anthracis. The toxin is composed of three proteins: protective antigen (PA), edema factor (EF), and lethal factor (LF). PA, by itself, is not toxic but rather plays the role of translocating EF and LF to a target cell's cytosol (Klimpel, K R et al., (1992) Proc Natl Acad Sci USA 89:10277-81; Molloy, S S et al. (1992) J Biol Chem 267:16396-402; Singh, Y et al., (1989) J. Biol. Chem. 264:11099-11102; Petosa, C et al., (1997) Nature 385:833-838). Two cell surface receptors for PA (anthrax toxin receptor or ANTXR) have recently been identified (Bradley, K A et al., (2001) Nature 414:225-9; Scobie, H M et al., (2003) Proc Natl Acad Sci USA 100:5170-4). Following binding to ANTXR, PA is cleaved by cell surface-associated furin, that removes a 20 kDa fragment and leaving a 63 kDa fragment PA₆₃) bound to ANTXR. This step is necessary to expose a binding site for EF or LF (Mogridge, J et al. (2002) Biochemistry 41:1079-82) as well as to remove steric hindrances to PA's subsequent oligomerization into a heptamer (Petosa et al., supra; Leppla, S H, In: Sourcebook of Bacterial Protein Toxins, Freer, A, ed., Academic Press, 1991, pp. 277-302; Milne, J C et al. (1994) J. Biol. Chem. 269:20607-12). After EF or LF binds to heptameric PA₆₃, the toxin complex is internalized via the endosomal pathway (Friedlander, A M (1986) J. Biol. Chem. 261:7123-26; Gordon, V M et al. (1988) Infec Immun 56:1066-9; Leppla, S H (1982) Proc Natl Acad Sci USA 79:3162-6). The acidic environment of the endosome induces a conformational change in the PA structure, causing it to form a pore through which EF or LF apparently transits into the cytosol.

EF is an adenylate cyclase (Leppla, supra). EF+PA (=edema toxin or EdTx) is not lethal but causes edema when injected subcutaneously (s.c.) (Beall, F A et al. (1962) J. Bacteriol. 83:1274-80; Stanley, J L et al. (1961) J. Gen. Microbiol. 26:49-66).

LF is a Zn²⁺-metalloprotease which specifically cleaves the NH₂-termini of several mitogen-activated protein kinase kinases (MAPKK=MEK, including MEKs 1, 2, 3, 4, 6 and 7) (Duesbery, N S et al. (1998) Science 280:734-7; Vitale, G et al. (1998) Biochem Biophys Res Commun 248:706-11; Pellizzari, R et al. (1999) FEBS Lett 462:199-204; Vitale, G et al. (2000) Biochem J 352 Pt 3:739-45). resulting in their inactivation (Duesbery et al., supra; Chopra, A P et al. (2003) J. Biol Chem 278:9402-6; Bardwell, A J et al. (2004) Biochem J 378:569-77). LF does not cleave MEK-5 (Vitale et al., supra). Although the combination of PA+LF (=lethal toxin or “LeTx”) does not cause edema, when injected intravenously (i.v.), it rapidlys induce hypotensive shock leading to death.

Mature LF is a large 776-amino-acid (90.2 kDa) protein (Bragg, T S et al. (1989) Gene 81:45-54). The full length protein shown below with the leader sequence present has 809 amino acids (SEQ ID NO:2). The crystal structure of LF has been solved to a resolution of 2.2 Å ((Pannifer, A D et al. (2001) Nature 414:229-33) and is depicted in FIG. 1. It is composed of four domains. Domain I comprises the NH₂-terminal portion, which binds PA. Domain II (residues 263-297 and 385-550) (based on the shorter, mature polypeptide; the residues defining of domain II in SEQ ID NO:1 are 296-330 and 418-583). These correspond to Domain IIa (SEQ ID NO:4) which is aa 296-375 of the LF protein (SEQ ID NO:2). Domain IIb aa sequence (SEQ ID NO:6) is from aa 419-583 of the LF protein. Domain II shows structural similarity with the adenosine diphosphate-ribosylating toxin of Bacillus cereus but lacks the residues required for nicotinamide adenine dinucleotide binding and catalysis. Domains III inserts into domain II and contains a series of four tandem imperfect repeats of a helix-turn element present in domain II. A previous report suggests this region is important for LF activity since deletion of the second imperfect repeat (residues 308-326 of the mature protein or residues 341-359 of SEQ ID NO:2) renders LF non-toxic (Arora, N & Leppla, S H (1993) J Biol Chem 268:3334-41). Acidic residues in domain III form specific contacts with the basic NH₂-termini of MEKs. Domain IV has limited structural homology to thermolysin and contains the catalytic core. Insertional mutagenesis within this domain (e.g., insertion of an Arg-Val dipeptide at residue 720) can eliminate LF's toxicity without blocking its ability to bind PA (Quinn, C P et al. (1991) J. Biol. Chem. 266:20124-30). Elements of domains II, III, and IV together create a long catalytic groove into which the NH₂-terminus of MEK fits, forming an active site complex.

The present inventor and his colleagues demonstrated the existence of an LF-interacting region (LFIR) located in C-terminal region of MEK1, adjacent to a proline-rich region where other regulatory molecules, including B-Raf, interact with MEK (Chopra, A P et al. (2003) J Biol Chem 278:9402-6). Mutation of conserved residues within this region prevented LF proteolysis of MEKs without altering MEK's kinase activity. The precise function of the LFIR is not certain, though it was hypothesized that it is required for MEK association with LF.

Again, MEKs are upstream activators of members of the MAPK family. These members comprise extracellular-signal-regulated kinases (ERKs) also known as mitogen-activated protein kinases (MAPKs), for example, ERK 1 or ERK 2 which are the same as MAPK 1 or MAPK 2). Seven different MEK enzymes have been described. MEKs 1 and 2 phosphorylate and activate ERK 1 and 2 (=MAPK 1 and 2) in response to activation by the ras pathway. MEKs 1 and 2 are stimulated by mitogens or growth factors. Mitogen-induced entry of cells into S-phase of the cell cycle is blocked by antisense ERK mRNA (Pages G et al., Proc Natl Acad Sci USA, 1993, 90:8319-23) dominant negative ERK mutants (Troppmair J et al., J Biol Chem, 1994, 269:7030-5; Frost J A et al., Proc Natl Acad Sci USA, 1994, 91:3844-8), and small molecule inhibitors of MEK1/2 such as PD98059 (Dudley D T et al., Proc Natl Acad Sci USA, 1995, 92:7686-7689) or PD184352 (Sebolt-Leopold J S et al., Nat Med, 1999, 5:810-6). MEKs also play a role in programmed cell death (see WO 02/076496, by the present inventor and colleagues, and references cited therein).

MEKs regulate cellular responses to mitogens as well as environmental stress. Inappropriate activation of these kinases contributes to tumorigenesis. Activated MAPK or elevated MAPK expression has been detected in a variety of human tumors including breast carcinoma and glioblastoma, as well as primary tumor cells derived from kidney, colon, and lung tissues (see, for example). MEK-ERK signalling has also been shown to play a critical role in tumor metastasis and in tumor angiogenesis (WO 02/076496, supra).

Abbreviations used; ATP, adenosine triphosphate; ANTXR, anthrax toxin receptor; COOH, carboxy; CHO, Chinese hamster ovary; df, degrees of freedom; EC₅₀, 50% effective concentration; EF, edema factor; ERK, extracellular regulated kinase; FPLC, fast pressure liquid chromatography; kDa, kilodalton; LeTx, lethal toxin; LF, lethal factor; LFIR, LF-interacting region; MEK, mitogen activated protein kinase kinase (MAPKK); NH₂, amino; p, probability; PA, protective antigen; SDS, sodium lauryl sulfate.

### SUMMARY OF THE INVENTION

The present inventor has discovered that a number of specific mutants of LF lose the ability or have reduced ability to bind to and interact productively with MEK-1 or MEK-2, the substrate of LF action. It is through proteolysis of MEK that the LF exerts its toxic effects.

Thus, the present invention is directed to a mutant or variant anthrax lethal factor (LF) polypeptide in which between one and five amino acid residues in domain II that is important for interaction with the LF substrates MEK-1 or MEK-2 (as well as MEKs-3, 4, 6 and 7), are either substituted, deleted, or chemically derivatized such that the polypeptide is inhibited compared to normal LF in binding to and interacting with said MEK, the residues selected from the group consisting of L293, K294, R491, L514 and N516. These position correspond to residues L326, K327, R524, L547 and N549 of SEQ ID NO:2.

In one embodiment of the mutant or variant LF, at least two amino acid residues in domain II is substituted or mutated, which two residues are selected from the group consisting of L514/L293, L514/K294 and L514/R491.

Preferably, in the above mutants or variants, one or more amino acid residues is substituted with Ala or Gly, most preferably with Ala.

A preferred group of mutants is L293A, K294A, R491A, L514A, and N516A, and double mutants L514A/L293A, L514A/K294A and L514A/R491A.

Also provided is a fragment of the above mutant or variant corresponding to domain IIa or domain IIb of LF, or a mixture of such fragments. Preferably, the sequence of the fragments is SEQ ID NO:4 or SEQ ID NO:6.

The present invention is directed to an isolated nucleic acid molecule that encodes the above mutant or variant LF polypeptide, or fragment. These nucleic acids may be used to produce the LF polypeptide or as immunogenic DNA vaccines by administration to a subject using methods and routes well-known in the art.

As a result of the recognition by the inventor that particular conformational epitopes of domain II of LF are rendered incapable or less capable of binding MEK as a result of the mutations in the above positions, it has become possible to design screening assays that examine the effect of a potential inhibitor of LF-MEK binding based solely on inhibition of binding (vs. inhibition of proteolysis which was the only disclosed basis for testing inhibitors prior to this invention. Any type of binding assay known in the art, using labeled or unlabeled components (LF, MEK) may be used. Other assays that are well-known for this purpose are those that do not require labels, such as with the BiaCore technology, or isothermal calorimetric assays, or an assay based on labeled reactants, the “AlphaScreen™ assay. Exemplified herein are assays that examine competition of B-Raf binding or in vitro MEK proteolysis (the latter of which, alone does not distinguish the binding phase from the proteolysis phase and would identify inhibitors of either or both phases).

The invention provides method for screening a test sample comprising an agent or compound being tested for its ability to inhibit the binding interaction of LF and MEK independent of any effect on LF-mediated proteolysis of MEK, comprising

(a) contacting a test sample with LF and a MEK protein; and
(b) assaying for the binding of LF to MEK;
(c) comparing the binding to the binding of LF in the absence of the test sample,
wherein, if the binding measured in (a) is lower than the binding measure in (b), the agent or compound is an inhibitor of LF-MEK binding. This method may further comprise the step of comparing the binding in step (b) with the binding to MEK of an LF mutant, variant or fragment as described herein.

The above method of claim may also comprise testing the ability of the sample to inhibit MEK proteolysis, wherein if the compound is positive in inhibiting the binding and negative in inhibiting the proteolysis, it is a pure binding inhibitor

Also provides is a method for screening a sample or multiplicity of samples comprising an agent or compound (or agents/compounds) being tested for (i) the ability to inhibit the binding interaction of LF and MEK and (ii) the ability to inhibit LF-mediated proteolysis of MEK and, comprising
(a) contacting a test sample with LF and a MEK protein,
(b) assaying for the binding of LF to MEK; and
(c) comparing the binding to the binding of LF in the absence of the test sample,
(d) independently of the assay of step (b), assaying for the proteolysis of MEK by LF in the presence of the test sample or samples, and
(e) comparing the proteolysis in (d) to the proteolysis of MEK by LF in the absence of the test sample,
   wherein, if the binding measured in (a) is lower than the binding measure in (b), and the proteolysis measured in (d) is lower that the proteolysis measure in (e) the agent or compound in the sample or the agents or compounds in the multiplicity of samples are inhibitors of LF-MEK binding and LF-mediated MEK proteolysis. This method may further comprise comparing the binding in step (b) with the binding to MEK of an LF mutant, variant or fragment as described herein.

The present invention includes an immunogenic or vaccine composition comprising: (a) the mutant or variant LF as above, and (b) an immunologically acceptable carrier or excipient. Also included are DNA vaccines, well-known in the art, that comprise (a) the nucleic acid molecule as above encoding the mutant of variant LF, and (b) an immunologically acceptable carrier or excipient.

The invention is further directed to a method of inducing LF-specific immunity in a subject comprising administering to the subject an immunogenically effective amount of the above polypeptide or nucleic acid immunogenic composition. The method can be used to generate LF-specific antibodies which may be stored, isolated, etc., and used in passive immunization.

### BRIEF DESCRIPTION OF THE DRAWINGS

FIG. 1. A surface plot of anthrax LF highlighting aliphatic residues. A space-filled surface plot of LF was generated using Protein Explorer® freeware. Aliphatic residues were identified and were found to fall into three clusters (labeled I, II, and III) adjacent to the catalytic groove. Residues are color-coded green for leucine, blue for isoleucine, and pink for valine. The NH₂-terminus of MEK is indicated in black.

FIG. 2. The toxicity of mutagenized LF. The toxicity of mutagenized LF was measured using macrophage lysis assays. The concentrations of LF protein containing a) alanine mutations of aliphatic residues in clusters I-III, and b) alanine mutations of residues located close to L514, that are required to cause a 50% maximal decrease in cell viability (the EC₅₀) was determined by interpolation and is presented as an average of at least three experiments, each of which was performed using independently purified batches of protein, plus and minus standard deviation between batches. The toxicity of double mutants was also measured using macrophage lysis assays (c) and is presented as an average of at least three experiments using independently purified batches of protein, plus and minus standard deviation between batches.

FIG. 3. The effects of point mutations upon LF functions. a) The effects of point mutations upon proteolytic activity of LF in macrophages was assessed by immunoblotting lysates of toxin-treated (2 h with 0.1 μg/ml PA, 0.01 μg/ml LF) J774A.1 cells with antibodies directed towards the NH₂-termini of MEK2 [MEK2 (NT)]. To control for loading and uniform protein expression, these blots were stripped and re-probed with antibodies directed towards the COOH-terminus of MEK2 [MEK2 (CT)]. Only wild-type LF and LF containing alanine mutations which had a neutral or marginal effect on toxicity were able to cleave MEK2. The results shown are representative of three experiments. (b) To test whether our mutant LF were able to bind PA [³⁵S] Met-labeled LF and LF mutants were incubated with CHO cells at 4° C., pH 7.0. After unbound protein was washed away, bound ³⁵S was quantitated using a liquid scintillation counter. LF (Y236A), which has been previously shown to be incapable of binding to PA (Lacy, D B et al. (2002) J Biol Chem 277:3006-10) was used as a negative control. The results shown are an average of at least three experiments and are expressed as a percentage of wild-type LF bound to cells ±standard deviation. (c) To test whether our mutant LF were able to translocate across a membrane [³⁵S] methionine-labeled LF and LF mutants were incubated with CHO cells at 4° C., pH 7.0. After unbound protein was washed away the cells were treated with low- or neutral-pH buffer. The low-pH buffer mimics the endosomal environment and triggers PA₆₃ pore formation and the subsequent translocation of LF to the cytosol. After this cells were treated with or without pronase to remove any surface-bound label, washed, lysed, and assayed for ³⁵S content. The results shown are an average of at least three experiments and are expressed as a percentage of label incorporated into cells that had not been treated with pronase, ±standard deviation.

FIGS. 4a-4d: Toxicity and proteolytic activity of purified LF and LF double mutants. FIG. 4a describes wild-type LF and selected LF double mutants were purified by fast pressure liquid chromatography and their toxicity was re-assessed using macrophage-cytotoxicity assays. J774A.1 cells were treated with PA plus varying concentrations of wild-type LF (x) and LF (L514A) as well as LF containing pairwise alanine mutations of L514 and N516, L514 and K294, or L514 and R491 as indicated in the methods section. Cell viability was assessed after 3 h treatment by AQ assay and is presented as an average of five experiments, plus and minus standard deviation.

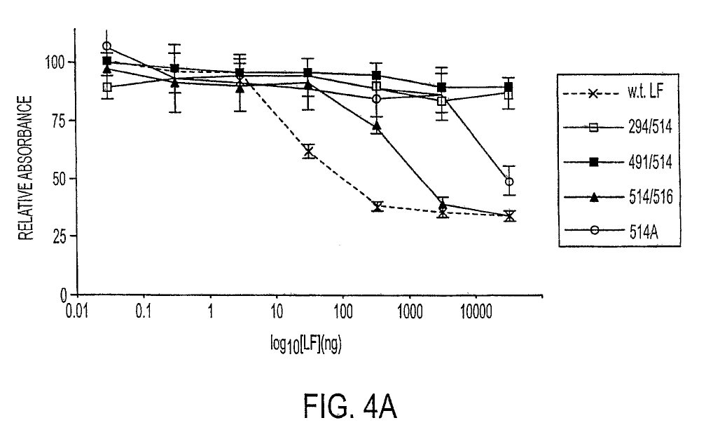

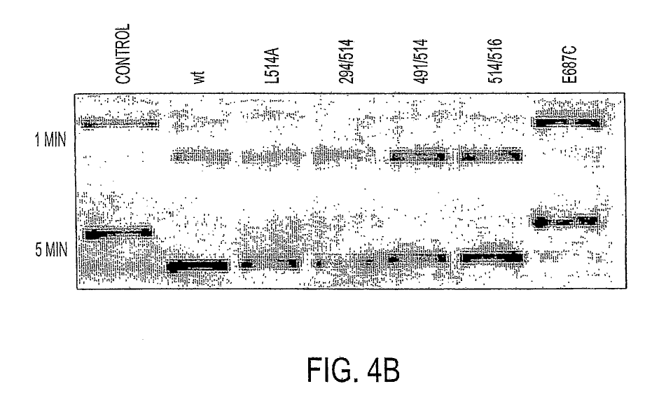

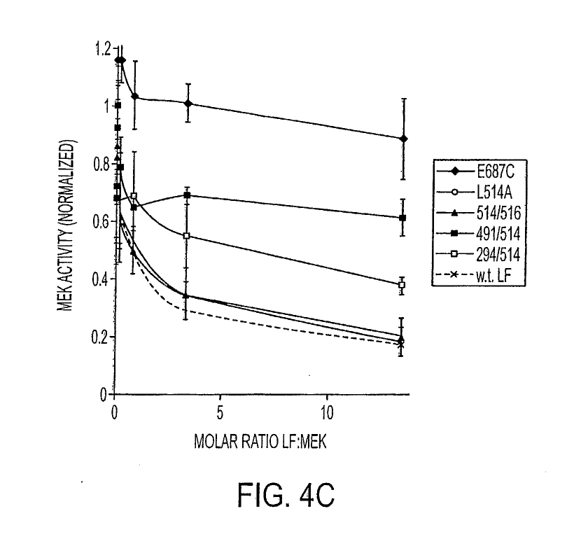

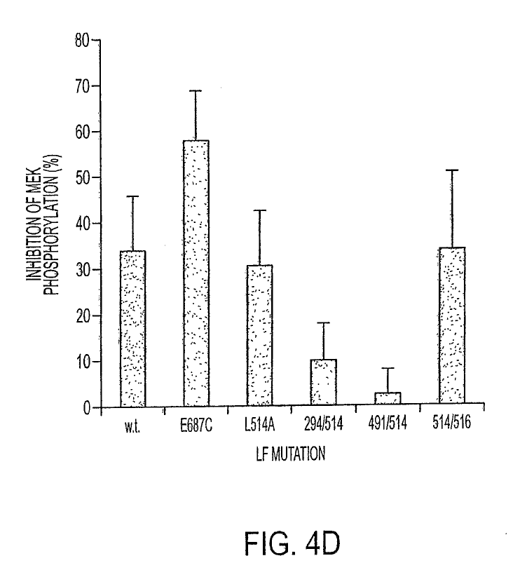

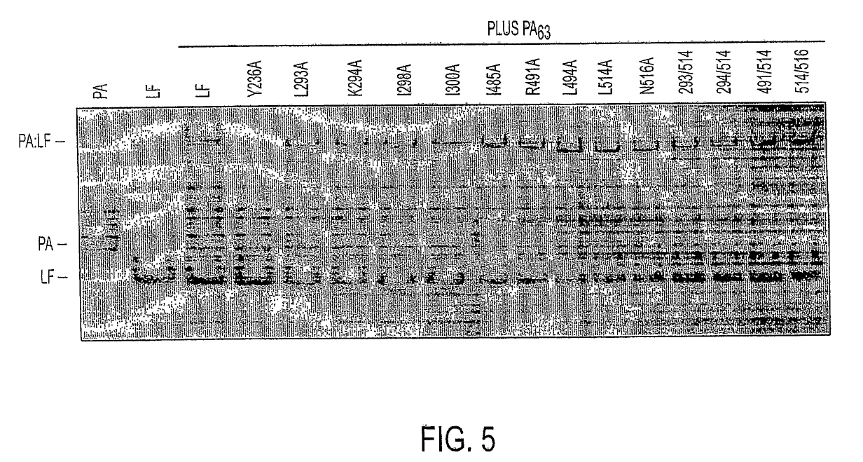

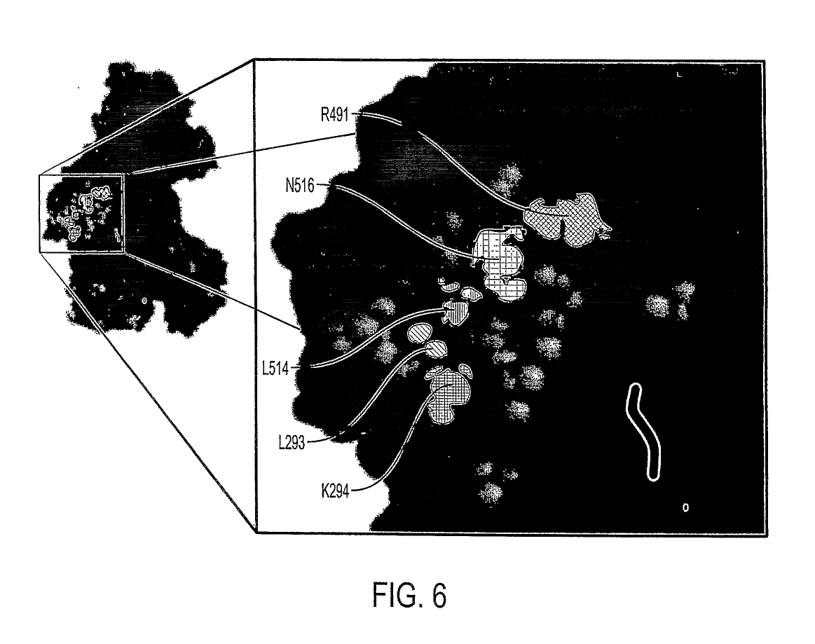

FIG. 4b shows results where His₆-tagged wild-type MEK1 (0.2 μg) was incubated with wild-type LF or LF mutants (0.2 μg) at 30° C.C for 1 or 5 min., proteins were separated by SDS-PAGE and immunoblotted with an antibody raised against residues 216-233 of human MEK1. MEK1 not reacted with LF (control) or reacted with inactive LF (E687C) are included as negative controls. MEK1 cleavage is indicated by increased electrophoretic mobility following proteolytic removal of the His₆-tag as well as the NH₂-terminus of MEK1.
FIG. 4c shows results of in vitro MEK proteolysis assays were performed in the presence of a constant concentration of MEK (0.35 μg) while varying the amount of LF (0.002 to 10 μg), using MEK activity (i.e. ERK phosphorylation) as a readout for LF activity. ERK phosphorylation was quantitated using a PhosphorImager. Ordinate; ERK phosphorylation normalized to control values obtained in the absence of LF in each experiment. Abscissa; the molar ratio of wild-type LF, LF (E687C), and LF (L514A) as well as LF containing pairwise alanine substitutions for L514 and N516, L514 and K294, or L514 and R491 to MEK1. The results are expressed as an average of at least three experiments, plus and minus standard deviation.
FIG. 4d shows studies of B-Raf phosphorylation of MEK in the presence of FPLC-purified LF and LF mutants assayed in vitro. MEK phosphorylation was quantitated using a PhosphorImager and normalized to MEK phosphorylation in the absence of LF. The results are expressed as an average of four experiments, plus and minus standard deviation.

FIG. 5 shows results of non-denaturing PA:LF gel-shift assays. To test whether the mutant LF retained the ability to bind PA, we performed non-denaturing gel-shift assays using LF and trypsin-nicked PA (PA₆₃). PA₆₃ was made by incubating 10 μl PA (7.3 mg/ml) with 1 μl trypsin (50 ng/μl) and 62 μl 10 mM Hepes (pH 8.0) for 15 min at room temperature. The trypsin was inactivated by the addition of 0.5 μl trypsin inhibitor (5 μg/ml, Type 1P from bovine pancreas, Sigma, St. Louis, Mo.). LF and LF mutant proteins (5 μg) were incubated with PA₆₃ (5 μg) for 15 min at room temperature. The samples were separated upon a 4-12% Tris-glycine gel following the addition of an equal volume of non-denaturing sample buffer (100 mM Tris-HCl, 10% glycerol, 0.0025% Bromophenol Blue pH 8.6) and using Tris-glycine non-denaturing running buffer (25 mM Tris-base, 192 mM glycine, pH 8.3). As a negative control we used LF containing an alanine substitution for tyrosine residue 236 (Y236A), which has been previously shown to be incapable of binding to PA (Park, S et al. (2000) Protein Expr Purif 18:293-302. Whereas wild-type LF as well as all mutant LF formed super-shifted complexes in the presence of PA₆₃, LF (Y236A) did not.

FIG. 6 is a surface plot of anthrax LF highlighting mutagenized residues. A space-filled surface plot of LF was generated using Protein Explorer® freeware. Residues identified as being critical for LF activity are colored yellow (K294), green (L293), red (L514), purple (N516), and orange (R491). Residues found to play a neutral or marginal role in LF activity are colored white. The NH₂-terminus of MEK is indicated in black. A magnified image of this region shows critical residues are organized side-by-side in a focused band (KLLNR) which lies at one end of the catalytic groove.

### DESCRIPTION OF THE PREFERRED EMBODIMENTS

Based on the conclusion that LF and MEK interact outside the active site complex (Chopra et al., supra), the present inventor conceived that LF must have a corresponding region in which the introduction of mutations at key residues should disrupt toxicity. The present invention identifies a cluster of residues in domain II of LF that play a key role in LF-mediated toxicity. Site directed mutagenesis was the preferred approach to achieve such identification. Once the existence of such a site or sites was known, and the existence of separate binding of LF to MEK, it opened the way to development of new screening methods that focus on inhibitors of this interactions, as distinct from the proteolysis function. This is different from the interactions of most proteases with their targets, where the binding and recognition functions all occur via the enzyme's catalytic/active site.

Ala substitution of the residues in this cluster substantially reduced LF toxicity and blocked proteolysis of MEK in cells. It is noteworthy that these residues are not contiguous in the primary sequence, but rather are a “sensitive” positions in the tertiary structure of the LF protein. In other words, the important regions of the molecule behave like “conformational” epitopes vs. linear epitopes.

Functional tests of these mutations indicated that loss of toxicity was not caused by interference with the ability of LF to bind PA, translocate across the membrane, or to cleave MEK in vitro. Rather, the loss of toxicity was related to a reduction in the ability of LF to interact with MEK.

The region containing this cluster of residues could is a useful therapeutic target for discovery and development of small molecule inhibitors that disrupt LF-MEK association and thereby block LF-mediated proteolysis of MEK, which would result in the inhibition of LF toxicity to cells. A drug discovered in this manner could be used to treat infections with natural or weaponized B. anthracis bacterial, or the impact of contact with isolated anthrax lethal toxin molecules.

In another embodiment, mutant LF molecules as described herein are useful as vaccine immunogens, because their administration to a subject to induce immunity to various protective epitopes of the molecule would not be accompanied by toxic effects of the LF.

The complete nucleotide and amino acid sequence of LF are shown below. The nucleotide sequence is SEQ ID NO:1 and the amino acid sequence is SEQ ID NO:2. This sequence is annotated by underscoring to show the nt and aa sequences corresponding to the two segment of domain II (which is made up of two regions, domains IIa and IIb). (The sequence between IIa and IIb is referred to as domain III, and, at the protein level, comprises a series of imperfect repeats of a motif found in domain II that together are considered to form a distinct region). Thus domain II comprises:

(a) the nt (SEQ ID NO:3 and aa (SEQ ID NO:4) sequence of domain IIa
(b) the nt (SEQ ID NO:5) and aa acid (SEQ ID NO:6) of domain IIb.

Domain IIa coding sequence (SEQ ID NO:3) is from nt 885-1125 of the LF DNA (SEQ ID NO:1).

Domain IIb coding sequence (SEQ ID NO:5) is from nt 1157-1749 of the LF DNA (SEQ ID NO:1).

Domain IIa aa sequence (SEQ ID NO:4) is from aa 296-375 of the LF protein (SEQ ID NO:2).

Domain IIb aa sequence (SEQ ID NO:6) is from aa 419-583 of the LF protein (SEQ ID NO:2).

The codons and amino acid residues that are bolded and italicized in domain II (

| | |
|---|---|
| atg aat ata aaa aaa gaa ttt ata aaa gta att | 60 |
| M N I K K E F I K V I | |
| agt atg tca tgt tta gta aca gca att | |
| S M S C L V T A I | |
| act ttg agt ggt ccc gtc ttt atc ccc ctt gta | 120 |
| T L S G P V F I P L V | |
| cag ggg gcg ggc ggt cat ggt gat gta | |
| Q G A G G H G D V | |
| ggt atg cac gta aaa gag aaa gag aaa aat aaa | 180 |
| G M H V K E K E K N K | |
| gat gag aat aag aga aaa gat gaa gaa | |
| D E N K R K D E E | |
| cga aat aaa aca cag gaa gag cat tta aag gaa | 240 |
| R N K T Q E E H L K E | |
| atc atg aaa cac att gta aaa ata gaa | |
| I M K H I V K I E | |
| gta aaa ggg gag gaa gct gtt aaa aaa gag gca | 300 |
| V K G E E A V K K E A | |
| gca gaa aag cta ctt gag aaa gta cca | |
| A E K L L E K V P | |
| tct gat gtt tta gag atg tat aaa gca att gga | 360 |
| S D V L E M Y K A I G | |
| gga aag ata tat att gtg gat ggt gat | |
| G K I Y I V D G D | |
| att aca aaa cat ata tct tta gaa gca tta tct | 420 |
| I T K H I S L E A L S | |
| gaa gat aag aaa aaa ata aaa gac att | |
| E D K K K I K D I | |
| tat ggg aaa gat gct tta tta cat gaa cat tat | 480 |
| Y G K D A L L H E H Y | |
| gta tat gca aaa gaa gga tat gaa ccc | |
| V Y A K E G Y E P | |
| gta ctt gta atc caa tct tcg gaa gat tat gta | 540 |
| V L V I Q S S E D Y V | |
| gaa aat act gaa aag gca ctg aac gtt | |
| E N T E K A L N V | |
| tat tat gaa ata ggt aag ata tta tca agg gat | 600 |
| Y Y E I G K I L S R D | |
| att tta agt aaa att aat caa cca tat | |
| I L S K I N Q P Y | |
| cag aaa ttt tta gat gta tta aat acc att aaa | 660 |
| Q K F L D V L N T I K | |
| aat gca tct gat tca gat gga caa gat | |
| N A S D S D G Q D | |
| ctt tta ttt act aat cag ctt aag gaa cat ccc | 720 |
| L L F T N Q L K E H P | |
| aca gac ttt tct gta gaa ttc ttg gaa | |
| T D F S V E F L E | |
| caa aat agc aat gag gta caa gaa gta ttt gcg | 780 |
| Q N S N E V Q E V F A | |
| aaa gct ttt gca tat tat atc gag cca | |
| K A F A Y Y I E P | |
| cag cat cgt gat gtt tta cag ctt tat gca ccg | 840 |
| Q H R D V L Q L Y A P | |
| gaa gct ttt aat tac atg gat aaa ttt | |
| E A F N Y M D K F | |
| aac gaa caa gaa ata aat cta tcc ttg gaa gaa | 900 |
| N E Q E I N L S L E E | |
| ctt aaa gat caa cgg atg ctg tca aga | |
| L K D Q R M L S R | |
| tat gaa aaa tgg gaa aag ata aaa cag cac tat | 960 |
| Y E K W E K I K Q H Y | |
| caa cac tgg agc gat tct tta tct gaa | |
| Q H W S D S L S E | |
| gaa gga aga gga ctt aag ctg cag att | 1020 |
| E G R G L L Q I | |
| cct att gag cca aag aaa gat gac ata | |
| P I E P K K D D I | |
| att cat tct tta tct caa gaa gaa aaa gag ctt | 1080 |
| I H S L S Q E E K E L | |
| cta aaa aga ata caa att gat agt agt | |
| L K R I Q I D S S | |
| gat ttt tta tct act gag gaa aaa gag ttt tta | 1140 |
| D F L S T E E K E F L | |
| aaa aag cta caa att gat att cgt gat | |
| K K L Q I D I R D | |
| tct tta tct gaa gaa gaa aaa gag ctt tta aat | 1200 |
| S L S E E E K E L L N | |
| aga ata cag gtg gat agt agt aat cct | |
| R I Q V D S S N P | |
| tta tct gaa aaa gaa aaa gag ttt tta aaa aag | 1260 |
| L S E K E K E F L K K | |
| ctg aaa ctt gat att caa cca tat gat | |
| L K L D I Q P Y D | |
| att aat caa agg ttg caa gat aca gga ggg tta | 1320 |
| I N Q R L Q D T G G L | |
| att gat agt ccg tca att aat ctt gat | |
| I D S P S I N L D | |
| gta aga aag cag tat aaa agg gat att caa aat | 1380 |
| V R K Q Y K R D I Q N | |
| att gat gct tta tta cat caa tcc att | |
| I D A L L H Q S I | |
| gga agt acc ttg tac aat aaa att tat ttg tat | 1440 |
| G S T L Y N K I Y L Y | |
| gaa aat atg aat atc aat aac ctt aca | |
| E N M N I N N L T | |
| gca acc cta ggt gcg gat tta gtt gat tcc act | 1500 |
| A T L G A D L V D S T | |
| gat aat act aaa att aat aga ggt att | |
| D N T K I N R G I | |
| ttc aat gaa ttc aaa aaa aat ttc aaa tat agt | 1560 |
| F N E F K K N F K Y S | |
| att tct agt aac tat atg att gtt gat | |
| I S S N Y M I V D | |
| ata aat gaa cct gca tta gat aat gag cgt | 1620 |
| I N E [symbol] P A L D N E R | |
| ttg aaa tgg aga atc caa tta tca cca | |
| L K W R I Q L S P | |
| gat act cga gca gga tat gaa gga aag | 1680 |
| D T R A G Y E G K | |
| ctt ata tta caa aga aac atc ggt ctg | |
| L I L Q R N I G L | |
| gaa ata aag gat gta caa ata att aag caa tcc | 1740 |
| E I K D V Q I I K Q S | |
| gaa aaa gaa tat ata agg att gat gcg | |
| E K E Y I R I D A | |
| aaa gta gtg cca aag agt aaa ata gat aca aaa | 1800 |
| K V V P K S K I D T K | |
| att caa gaa gca cag tta aat ata aat | |
| I Q E A Q L N I N | |
| cag gaa tgg aat aaa gca tta ggg tta cca aaa | 1860 |
| Q E W N K A L G L P K | |
| tat aca aag ctt att aca ttc aac gtg | |
| Y T K L I T F N V | |
| cat aat aga tat gca tcc aat att gta gaa agt | 1920 |
| H N R Y A S N I V E S | |
| gct tat tta ata ttg aat gaa tgg aaa | |
| A Y L I L N E W K | |
| aat aat att caa agt gat ctt ata aaa aag gta | 1980 |
| N N I Q S D L I K K V | |
| aca aat tac tta gtt gat ggt aat gga | |
| T N Y L V D G N G | |
| aga ttt gtt ttt acc gat att act ctc cct aat | 2040 |
| R F V F T D I T L P N | |
| ata gct gaa caa tat aca cat caa gat | |
| I A E Q Y T H Q D | |
| gag ata tat gag caa gtt cat tca aaa ggg tta | 2100 |
| E I Y E Q V H S K G L | |
| tat gtt cca gaa tcc cgt tct ata tta | |
| Y V P E S R S I L | |
| ctc cat gga cct tca aaa ggt gta gaa tta agg | 2160 |
| L H G P S K G V E L R | |
| aat gat agt gag ggt ttt ata cac gaa | |
| N D S E G F I H E | |
| ttt gga cat gct gtg gat gat tat gct gga tat | 2220 |
| F G H A V D D Y A G Y | |
| cta tta gat aag aac caa tct gat tta | |
| L L D K N Q S D L | |
| gtt aca aat tct aaa aaa ttc att gat att ttt | 2280 |
| V T N S K K F I D I F | |
| aag gaa gaa ggg agt aat tta act tcg | |
| K E E G S N L T S | |
| tat ggg aga aca aat gaa gcg gaa ttt ttt gca | 2340 |
| Y G R T N E A E F F A | |
| gaa gcc ttt agg tta atg cat tct acg | |
| E A F R L M H S T | |
| gac cat gct gaa cgt tta aaa gtt caa aaa aat | 2400 |
| D H A E R L K V Q K N | |
| gct ccg aaa act ttc caa ttt att aac | |
| A P K T F Q F I N | |
| gat cag att aag ttc att att aac tca taa | 2427 |
| D Q I K F I I N S - | |

The separate amino acid sequence of LF (SEQ ID NO:2) is shown separately below and is annotated as follows:

| | |
|---|---|
| MNIKKEFIKV ISMSCLVTAI TLSGPVFIPL VQGAGGHGDV | 50 |
| GMHVKEKEKN | |
| KDENKRKDEE RNKTQEEHLK ETMKHIVKIE VKGEEAVKKE | 100 |
| AAEKLLEKVP | |
| SDVLEMYKAI GGKIYIVDGD ITKHISLEAL SEDKKKIKDI | 150 |
| YGKDALLHEH | |
| YVYAKEGYEP VLVTQSSEDY VENTEKALNV YYEIGKILSR | 200 |
| DILSKTNQPY | |
| QKFLDVLNTI KNASDSDGQD LLFTNQLKEH PTDFSVEFLE | 250 |
| QNSNEVQEVF | |
| AKAFAYYIEP QHRDVLQLYA PEAFNYMDKF NEQEINLSLE | 300 |
| ELKDQRMLSR | |
| YEKWEKIKQH YQHWSDSLSE EGRGL KLQ IPIEPKKDDI | 350 |
| IHSLSQEEKE | |
| LLKRIQIDSS DFLSTEEKEF LKKLQIDIRD SLSEEEKELL | 400 |
| NRIQVDSSNP | |
| LSEKEKEFLK KLKLDIQPYD INQRLQDTGG LIDSPSINLD | 450 |
| VRKQYKRDIQ | |
| NIDALLHQSI GSTLYNKIYL YENMNINNLT ATLGADLVDS | 500 |
| TDNTKINRGI | |
| FNEFKKNFKY STSSNYMIVD INE PALDNE RLKWRIQLSP | 550 |
| DTRAGY E G | |
| KLILQRNIGL EIKDVQIIKQ SEKEYIRIDA KVVPKSKIDT | 600 |
| KIQEAQLNIN | |
| QEWNKALGLP KYTKLITFNV HNRYASNIVE SAYLILNEWK | 650 |
| NNIQSDLIKK | |
| VTNYLVDGNG RFVFTDITLP NIAEQYTHQD ETYEQVHSKG | 700 |
| LYVPESRSIL | |
| LHGPSKGVEL RNDSEGFIHE FGHAVDDYAG YLLDKNQSDL | 750 |
| VTNSKKFIDI | |
| FKEEGSNLTS YGRTNEAEFF AEAFRLMHST DHAERLKVQK | 800 |
| NAPKTFQFIN | |
| DQIKFIINS | 809 |

Below, the nucleotide and amino acid sequences of domain IIa and IIb are shown “removed” from the full length LF sequences above (with the annotations of codons/amino acids of particular importance to this invention still annotated as bold and italic.

(1) Domain IIa polypeptide (SEQ ID NO: 4):
RMLSRYEKWE KIKQHYQHWS DSLSEEGRGL [symbol] KLQIPIEP
KKDDIIHSLS QEEKELLKRI QTDSSDFLST EEKEFLKKLQ
(2) Domain IIb polypeptide (SEQ ID NO: 6)
DINQRLQDTG GLIDSPSINL DVRKQYKRDI QNIDALLHQS
IGSTLYNKIY LYENMNINNL TATLGADLVD STDNTKINRG
IFNEFKKNFK YSISSNYMIV DINE [symbol] PALDN ERLKWRIQLS
PDTRAGY E [symbol] GKLILQRNIG LEIKDVQIIK QSEKEYIRID AKVV
(3) Domain IIIa coding seguence (SEQ ID NO: 3):
cgg atg ctg tca aga tat gaa aaa tgg gaa aag ata
aaa cag cac tat caa cac tgg agc gat tct tta tct
gaa gaa gga aga gga ctt [symbol] aag ctg cag att
cct att gag cca aag aaa gat gac ata att cat tct
tta tct caa gaa gaa aaa gag ctt cta aaa aga ata
caa att gat agt agt gat ttt tta tct act gag gaa
aaa gag ttt tta aaa aag cta caa
(4) Domain IIb coding sequence (SEQ ID NO: 5):
gat att aat caa agg ttg caa gat aca gga ggg tta
att gat agt ccg tca att aat ctt gat gta aga aag
cag tat aaa agg gat att caa aat att gat gct tta
tta cat caa tcc att gga agt acc ttg tac aat aaa
att tat ttg tat gaa aat atg aat atc aat aac ctt
aca gca acc cta ggt gcg gat tta gtt gat tcc act
gat aat act aaa att aat aga ggt att ttc aat gaa
ttc aaa aaa aat ttc aaa tat agt att tct agt aac
tat atg att gtt gat ata aat gaa [symbol] cct gca tta
gat aat gag cgt ttg aaa tgg aga atc caa tta tca
cca gat act cga gca gga tat [symbol] gaa [symbol] gga aag
ctt ata tta caa aga aac atc ggt ctg gaa ata aag
gat gta caa ata att aag caa tcc gaa aaa gaa tat
ata agg att gat gcg aaa gta gtg

In the following sections, the positions to be mutated were described using a somewhat modified numbering system than above, in which the first 33 residues of SEQ ID NO:2 were omitted from numbering as they represent the leader sequence of the polypeptide (shown as double underscored above. Thus, the following list shows the equivalent (identical) amino acid residues

| Residue # as shown | Residue # in |
|---|---|
| in SEQ ID NO: 2 | shortened sequence* |
| L326 | L293 |
| K327 | K294 |
| R524 | R491 |
| L547 | L514 |
| N549 | N516 |

*from which 33 residues of leader sequence was subtracted

Thus, all references to L293, K294, R491, L514 or N516 and mutations at those sites in this application, particularly in the Examples, refer to positions L326, K327, R524, L547 and N549, respectively, of SEQ ID NO:2. Of course, and their coding sequences of SEQ ID NO:1)

Preferred mutants are amino acid substitution variants at L293, K 294, R491, N516; any combination thereof is also intended, with the combinations of L514/L293, L514/K294 and L514/491 preferred. Preferred substitution is with Ala or Gly. Other substitutions can be made in accordance with this invention. Similarly, deletion of any one, two, three, four or all five of these residues are also included in the scope of this invention.

It would be a matter of routine testing to make such substitutions or deletions and test them using the methods described herein, to determine which results in a polypeptide having the desired property, namely an LF molecule with a reduced or no ability (within the limits of the testing systems) to interact productively with MEK, such that these LF mutants have reduced or no toxicity.

The polypeptides of the present invention including not only full length LF molecules that comprise the domain II mutations described herein, but also shorter molecules, such as domain II peptides themselves that include one or more of the mutations described herein. Preferred examples are mutated forms of SEQ ID NO:4 and SEQ ID NO:6 which can be use in the screening assays (of inhibition of binding) described below, alone or in combination, in place of the full length LF molecules.

### Chemical Derivatives of Anthrax LF

In addition the mutants and variants described herein, the present invention includes LF polypeptides in which have been chemically modified or derivatized

Covalent modifications of the LF polypeptides may be introduced by reacting targeted amino acid residues with an organic derivatizing agent that is capable of reacting with selected side chains or terminal residues. The preferred derivatives are those that mimic the mutations by inhibiting the ability of the LF chemical derivative to bind to and productively interact with MEK leading to MEK proteolysis.

For examples lysinyl and amino terminal residues are reacted with succinic or other carboxylic acid anhydrides. Derivatization with these agents reverses the charge of the lysinyl residues. Other suitable reagents for derivatizing α-amino-containing residues include imidoesters such as methylpicolinimidate; pyridoxal phosphate; pyridoxal; chloroborohydride; trinitrobenzenesulfonic acid; O-methylisourea; 2,4 pentanedione; and transaminase-catalyzed reaction with glyoxylate.

Arginyl residues are modified by reaction with one or several conventional reagents, including phenylglyoxal, 2,3-butanedione, 1,2-cyclohexanedione, and ninhydrin. Such derivatization requires that the reaction be performed in alkaline conditions because of the high pKₐ of the guanidine functional group. Furthermore, these reagents may react with the groups of lysine as well as the arginine ε-amino group.

Another type of chemical derivative is one in which a mutant LF of the present invention is further derivatized in order to improve its immunogenicity when used as a vaccine composition. Such derivatization are used to cross-link the polypeptide to itself (to make conjugates with improved immunogenic properties as is known in the art) or to various water-insoluble support matrices or other macromolecular carriers. Carboxyl side groups (aspartyl or glutamyl) are selectively modified by reaction with carbodiimides (R′—N—C—N—R′) such as 1-cyclohexyl-3-(2-morpholinyl-(4-ethyl) carbodiimide or 1-ethyl-3-(4-azonia-4,4-dimethylpentyl) carbodiimide. Commonly used cross-linking agents include 1,1-bis(diazoacetyl)-2-phenylethane, glutaraldehyde, N-hydroxysuccinimide esters, esters with 4-azidosalicylic acid, homobifunctional imidoesters, including disuccinimidyl esters such as 3,3′-dithiobis(succinimidylpropionate), and bifunctional maleimides such as bis-N-maleimido-1,8-octane.

Derivatizing agents such as methyl-3-[(p-azidophenyl)dithio]propioimidate yield photoactivatable intermediates that are capable of forming crosslinks in the presence of light. Alternatively, reactive water-insoluble matrices such as cyanogen bromide-activated carbohydrates and the reactive substrates described in U.S. Pat. Nos. 3,969,287; 3,691,016; 4,195,128; 4,247,642; 4,229,537; and 4,330,440 are employed for protein immobilization.

### Method of Screening for Inhibitors of LF/MEK Interaction

These clustered residues define a surface epitope of LF in domain II which is required for LF toxicity. Small molecules which occlude this site can thus serve as LF inhibitors and be used as drugs to treat the effects of B. anthracis infection or other effects of Anthrax lethal toxin in a subject. Thus, one embodiment of the present invention is a method to identify such inhibitors of LF-MEK binding/interaction. This method involves incubating a test or candidate molecule or agent with LF and MEK and measuring the ability of the candidate molecule/agent to prevent binding of MEK—using any binding assay. Alternatively, it is possible to assay for the inhibition of the cleavage of MEK and independently assaying for inhibition of LF-mediated proteolysis, such that inhibitors can be found that inhibit binding only, proteolysis only, or both, depending how the screening assays are combined.

Methods described in the Examples below and in the references cited herein provide certain appropriate techniques to use for such measurements. These techniques are considered conventional and routine in the art and need not be detailed here any further. Others are well-known in the art. Duesbery et al., U.S. Pat. No. 6,485,925 describes a method for evaluating agents for their ability to inhibit LF proteolysis of MEK. However, prior to the making of the present invention, there was no basis for evaluating a compounds ability to inhibit LF/MEK interactions at a stage prior to the proteolysis step.

### Pharmaceutical and Immunogenic Compositions

The present invention thus includes a “pharmaceutical” or “immunogenic” composition comprising a domain II mutant of LF as described, or a chemical derivative, analogue, or mimetic thereof, along with a pharmaceutically or immunologically acceptable excipient. Thus, the term “therapeutic composition” includes immunogenic or vaccine compositions and any other pharmaceutical comprising the LF mutant polypeptide, derivative, analogue, or mimetic (or nucleic acid if a DNA vaccine composition is to be used) and a therapeutically acceptable carrier or excipient. General methods to prepare immunogenic or vaccine compositions are described in Remington's Pharmaceutical Science; Mack Publishing Company Easton, Pa. (latest edition).

The invention provides a method of treating a subject, preferably a human, by immunizing or vaccinating the subject to induce an antibody response and any other accompanying protective form of immune reactivity against anthrax LF or lethal toxin.

The immunogenic material may be adsorbed to or conjugated to beads such as latex or gold beads, ISCOMs, and the like. Immunogenic compositions may comprise adjuvants, which are substance that can be added to an immunogen or to a vaccine formulation to enhance the immune-stimulating properties of the immunogenic moiety. Liposomes are also considered to be adjuvants (Gregoriades, G. et al., Immunological Adjuvants and Vaccines, Plenum Press, New York, 1989) Examples of adjuvants or agents that may add to the effectiveness of proteineaceous immunogens include aluminum hydroxide, aluminum phosphate, aluminum potassium sulfate (alum), beryllium sulfate, silica, kaolin, carbon, water-in-oil emulsions, and oil-in-water emulsions. A preferred type of adjuvant is muramyl dipeptide (MDP) and various MDP derivatives and formulations, e.g., N-acetyl-D-glucosaminyl-(β1-4)-N-acetylmuramyl-L-alanyl-D-isoglutamine (GMDP) (Hornung, R L et al. Ther Immunol 1995 2:7-14) or ISAF-l (5% squalene, 2.5% pluronic L121, 0.2% Tween 80 in phosphate-buffered solution with 0.4 mg of threonyl-muramyl dipeptide; see Kwak, L W et al. (1992) N. Engl. J. Med., 327: 1209-38). Other useful adjuvants are, or are based on, bacterial endotoxin, lipid X, whole organisms or subcellular fractions of the bacteria Propionobacterium acnes or Bordetella pertussis, polyribonucleotides, sodium alginate, lanolin, lysolecithin, vitamin A, saponin and saponin derivatives such as QS21 (White, A. C. et al. (1991) Adv. Exp. Med. Biol., 303:207-210) which is now in use in the clinic (Helling, F et al. (1995) Cancer Res., 55:2783-88; Davis, T A et al. (1997) Blood, 90:509). QS21 is a triterpene glycoside from the South American tree Quillaja saponaria (Soltysik S et al., 1993, Ann N Y Acad Sci 690:392-5). Other adjuvants include levamisole, DEAE-dextran, blocked copolymers or other synthetic adjuvants. A number of other adjuvants are available commercially from various sources, for example

### (a) Merck Adjuvant 65 (Merck and Company, Inc., Rahway, N.J.)

### (b) Freund's Complete or Incomplete Adjuvant (Difco Laboratories, Detroit, Mich.),

(c) Alhydrogel®-aluminum hydroxide gel, one of the oldest adjuvants known and approved for humans
(d) Amphigen (which may not be a registered trademark) and is an oil in water preparation defined in more detail in, for example, US Pat Publication 20050058667A1 (Mar. 17, 2005) which further cites U.S. Pat. No. 5,084,269 which states that “AMPHIGEN™ consists of de-oiled lecithin dissolved in an oil, usually light liquid paraffin.”. Veterinary Practice (at the world-wide web URL of .“vpmag.co.uk/news/article.php?article=1483020736.html”) refers to it as consisting of oil micelles coated with lecithin; or
(e) a mixture of Amphigen and Alhydrogel®.

A preferred effective dose for treating a subject in need of the present treatment, preferably a human, is an amount of up to about 100 milligrams of active compound per kilogram of body weight. A typical single dosage of the peptide, chimeric protein or peptidomimetic is between about 1 ng and about 100 mg/kg body weight, and preferably from about 10 μg to about 50 mg/kg body weight. A total daily dosage in the range of about 0.1 milligrams to about 7 grams is preferred for intravenous administration. A useful dose of an antibody for passive immunization is between 10-100 mg/kg. These dosages can be determined empirically in conjunction with the present disclosure and state-of-the-art.

The foregoing ranges are, however, suggestive, as the number of variables in an individual treatment regime is large, and considerable excursions from these preferred values are expected. As is evident to those skilled in the art, the dosage of an immunogenic composition may be higher than the dosage of the compound used to treat. Not only the effective dose but also the effective frequency of administration is determined by the intended use, and can be established by those of skill without undue experimentation. The total dose required for each treatment may be administered by multiple doses or in a single dose.

Pharmaceutically acceptable acid addition salts of certain compounds of the invention containing a basic group are formed where appropriate with strong or moderately strong, non-toxic, organic or inorganic acids by methods known to the art. Exemplary of the acid addition salts that are included in this invention are maleate, fumarate, lactate, oxalate, methanesulfonate, ethanesulfonate, benzenesulfonate, tartrate, citrate, hydrochloride, hydrobromide, sulfate, phosphate and nitrate salts. Pharmaceutically acceptable base addition salts of compounds of the invention containing an acidic group are prepared by known methods from organic and inorganic bases and include, for example, nontoxic alkali metal and alkaline earth bases, such as calcium, sodium, potassium and ammonium hydroxide; and nontoxic organic bases such as triethylamine, butylamine, piperazine, and tri(hydroxymethyl)methylamine.

The compounds of the invention, as well as the pharmaceutically acceptable salts thereof, may be incorporated into convenient dosage forms, such as capsules, impregnated wafers, tablets or preferably injectable preparations. Solid or liquid pharmaceutically acceptable carriers may be employed.

Preferably, the compounds of the invention are administered systemically, e.g., by injection or infusion. Administration may be by any known route, preferably intravenous, subcutaneous, intramuscular, intrathecal, intracerebroventricular, or intraperitoneal. (Other routes are noted below) Injectables can be prepared in conventional forms, either as solutions or suspensions, solid forms suitable for solution or suspension in liquid prior to injection, or as emulsions.

To enhance delivery or immunogenic activity, the compound can be incorporated into liposomes using methods and compounds known in the art.

DNA immunogens are administered via gene gun, or by injection intramuscularly or subcutaneously as is well-known in the art.

The pharmaceutical preparations are made following conventional techniques of pharmaceutical chemistry. The pharmaceutical compositions may also contain minor amounts of nontoxic auxiliary substances such as wetting or emulsifying agents, pH buffering agents and so forth. The peptides are formulated using conventional pharmaceutically acceptable parenteral vehicles for administration by injection. These vehicles are nontoxic and therapeutic, and a number of formulations are set forth in Remington's Pharmaceutical Sciences, Gennaro, 18th ed., Mack Publishing Co., Easton, Pa. (1990)). Nonlimiting examples of excipients are water, saline, Ringer's solution, dextrose solution and Hank's balanced salt solution. Formulations according to the invention may also contain minor amounts of additives such as substances that maintain isotonicity, physiological pH, and stability. In addition, suspensions of the active compounds as appropriate oily injection suspensions may be administered. Suitable lipophilic solvents or vehicles include fatty oils, for example, sesame oil, or synthetic fatty acid esters, for example, ethyl oleate or triglycerides. Aqueous injection suspensions may contain substances which increase the viscosity of the suspension. Optionally, a suspension may contain stabilizers.

The polypeptides nucleic acids and other useful compositions of the invention are preferably formulated in purified form substantially free of aggregates and other protein materials, preferably at concentrations of about 1.0 ng/ml to 100 mg/ml.

Having now generally described the invention, the same will be more readily understood through reference to the following examples which are provided by way of illustration, and are not intended to be limiting of the present invention, unless specified.

### EXAMPLE I

### Methods and Materials

### Cell Lines, Culture and Reagents

The murine macrophage-derived J774A.1 and the Chinese hamster ovarian epithelial (CHO)-K1 cell lines were obtained from the ATCC (Manassas, Va.). J774A.1 cells were cultured in Dulbecco's Modified Eagle Medium (DMEM) supplemented with 10% fetal bovine serum (FBS), 1% penicillin/streptomycin. CHO-K1 were cultured in Ham's F-12 medium supplemented with 10% FBS, and 1% penicillin/streptomycin. Both cell-lines were maintained at 37° C. in a humidified 5% CO₂ incubator.

### Site-Directed Mutagenesis

Alanine-substitutions in LF were generated by introducing mutations into a B. anthracis LF expression vector pSJ115 (Park et al., supra) with the use of the Quickchange™ site-directed mutagenesis kit (Stratagene, La Jolla, Calif.) following manufacturer's instructions except that primer extension was allowed to continue for 18 min and the deoxynucleotide triphosphate (dNTP) stocks were modified to reflect the high deoxyadenylate and deoxythymidylate content (70%) of LF the gene (Bragg et al., supra).

The primers used for site-directed mutagenesis are listed in Table 1 below:

TABLE 1

Primers used to generate alanine substitutions in LF.

| | | | |
|---|---|---|---|
| | | | SEQ |
| | Primer | | ID |
| Residue | # | primer sequence | NO: |

| | | | |
|---|---|---|---|
| Trp²⁷¹ | 1 | 5′-GTCAAGATATGAAAAAGCGGAAAAGATAAAACAG-3′ | 7 |
| | 2 | 5′-CTGTTTTATCTTTTCCGCTTTTTCATATCTTGAC-3′ | 8 |
| Trp²⁸¹ | 1 | 5′-GCACTATCAACACGCGAGCGATTCTTTATCTGAA-3′ | 9 |
| | 2 | 5′-TTCAGATAAAGAATCGCTCGCGTGTTGATAGTGC-3′ | 10 |
| Leu²⁸⁵ | 1 | 5′-GTGGAGCGATTCTGCGTCTGAAGAAGGAAGAGGA-3′ | 11 |
| | 2 | 5′-TCCTCTTCCTTCTTCAGACGCAGAATCGCTCCAC-3′ | 12 |
| Arg²⁹⁰ | 1 | 5′-GAAGAAGGAGCG GGACTTTTAAAAAAGCTG-3′ | 13 |
| | 2 | 5′-CTTCTTCCTCGCCCTGAAAATTTTTTCGAC-3′ | 14 |
| Leu²⁹³ | 1 | 5′-GAAGGAAGAGGACTTGCGAAAAAGCTGCAGATT-3′ | 15 |
| | 2 | 5′-AATCTGCAGCTTTTTCGCAAGTCCTCTTCCTTC-3′ | 16 |
| Lys²⁹⁴ | 1 | 5′-GAAGGAAGAGGACTTTTAGCAAAGCTGCAGATT-3′ | 17 |
| | 2 | 5′-AATCTGCAGCTTTGCTAAAAGTCCTCTTCCTTC-3′ | 18 |
| Gln²⁹⁷ | 1 | 5′-GGACTTTTAAAAAAGCTGGCAATTCCTATTGAG-3′ | 19 |
| | 2 | 5′-CTCAATAGGAATTGCCAGCTTTTTTAAAAGTCC-3′ | 20 |
| Ile²⁹⁸ | 1 | 5′-GGACTTTTAAAAAAGCTGCAGGCACCTATTGAGCCAAAG-3′ | 21 |
| | 2 | 5′-CTTTGGCTCAATAGGTGCCTGCAGCTTTTTTAAAAGTCC-3′ | 22 |
| Ile³⁰⁰ | 1 | 5′-GCTGCAGATTCCTGCGGAGCCAAAGAAAGAT-3′ | 23 |
| | 2 | 5′-ATCTTTCTTTGGCTCCGCAGGAATCTGCAGC-3′ | 24 |
| Ile³²² | 1 | 5′-GAGCTTCTAAAAAGAGCACAAATTGATAGTAGTGAT-3′ | 25 |
| | 2 | 5′-ATCACTACTATCAATTTGTGCTCTTTTTAGAAGCTC-3′ | 26 |
| Ile³⁴³ | 1 | 5′-GAGTTTTTAAAAAAGCTACAAGCAGATATTCGTGATTCT-3′ | 27 |
| | 2 | 5′-AGAATCACGAATATCTGCTTGTAGCTTTTTTAAAAACTC-3′ | 28 |
| Leu³⁴⁹ | 1 | 5′-GATATTCGTGATTCTGCATCTGAAGAAGAAAAAGAG-3′ | 29 |
| | 2 | 5′-CTCTTTTTCTTCTTCAGATGCAGAATCACGAATATC-3′ | 30 |
| Leu³⁵⁷ | 1 | 5′-GAAAAAGAGCTTGCAAATAGAATACAGGTGGAT-3′ | 31 |
| | 2 | 5′-ATCCACCTGTATTCTATTTGCAAGCTCTTTTTC-3′ | 32 |
| Val³⁶² | 1 | 5′-GAGCTTTTAAATAGAATACAGGCAGATAGTAGTAATCCT-3′ | 33 |
| | 2 | 5′-AGGATTACTACTATCTGCCTGTATTCTATTTAAAAGCTC-3′ | 34 |
| Leu⁴⁵⁰ | 1 | 5′-GAATATCAATAACCTTACAGCAACCGCAGGTGCGGAT-3′ | 35 |
| | 2 | 5′-ATCCGCACCTGCGGTTGCTGTAAGGTTATTGATATTC-3′ | 36 |
| Ile⁴⁶⁷ | 1 | 5′-GATAATACTAAAATTAATAGAGGTGCATTCAATGAA-3′ | 37 |
| | 2 | 5′-TTCATTGAATGCACCTCTATTAATTTTAGTATTATC-3′ | 38 |
| Ile⁴⁸⁵ | 1 | 5′-GAGTATTTCTAGTAACTATATGGCAGTTGATATAAAT-3′ | 39 |
| | 2 | 5′-ATTTATATCAACTGCCATATAGTTACTAGAAATACTC-3′ | 40 |
| Arg⁴⁹¹ | 1 | 5′-GATATAAATGAAGCGCCTGCATTAGATAATGAG-3′ | 41 |
| | 2 | 5′-CTCATTATCTAATGCAGGCGCTTCATTTATATC-3′ | 42 |
| Leu⁴⁹⁴ | 1 | 5′-GATATAAATGAAAGGCCTGCAGCAGATAATGAGCGT-3′ | 43 |
| | 2 | 5′-ACGCTCATTATCTGCTGCAGGCCTTTCATTTATATC-3′ | 44 |
| Tyr⁵¹³ | 1 | 5′-GATACTCGAGCAGGAGCGTTAGAAAATGGAAAGCTT-3′ | 45 |
| | 2 | 5′-AAGCTTTCCATTTTCTAACGCTCCTGCTCGAGTATC-3′ | 46 |
| Leu⁵¹⁴ | 1 | 5′-GATACTCGAGCAGGATATGCGGAAAATGGAAAGCTT-3′ | 47 |
| | 2 | 5′-AAGCTTTCCATTTTCCGCATATCCTGCTCGAGTATC-3′ | 48 |
| Glu⁵¹⁵ | 1 | 5′-GATACTCGAGCAGGATATTTAGCGAATGGAAAGCTT-3′ | 49 |
| | 2 | 5′-AAGCTTTCCATTCGCTAAATATCCTGCTCGAGTATC-3′ | 50 |
| Asn⁵¹⁶ | 1 | 5′-GATACTCGAGCAGGATATTTAGAAGCGGGAAAGCTT-3′ | 51 |
| | 2 | 5′-AAGCTTTCCCGCTTCTAAATATCCTGCTCGAGTATC-3′ | 52 |
| Lys⁵¹⁸ | 1 | 5′-GCAGGATATTTAGAAAATGGAGCGCTTATATTACAA-3′ | 53 |
| | 2 | 5′-TTGTAATATAAGCGCTCCATTTTCTAAATATCCTGC-3′ | 54 |
| Leu⁶⁷⁷ | 1 | 5′-GGACCTTCAAAAGGTGTAGAAGCAAGGAATGATAGTGAG-3′ | 55 |
| | 2 | 5′-CTCACTATCATTCCTTGCTTCTACACCTTTTGAAGGTCC-3′ | 56 |
| Leu⁷²⁵ | 1 | 5′-GAAGGGAGTAATGCAACTTCGTATGGGAGAACAAAT-3′ | 57 |
| | 2 | 5′-ATTTGTTCTCCCATACGAAGTTGCATTACTCCCTTC-3′ | 58 |
| Leu⁷⁴³ | 1 | 5′-GCAGAAGCCTTTAGGGCGATGCATTCTACGGACCAT-3′ | 59 |
| | 2 | 5′-ATGGTCCGTAGAATGCATCGCCCTAAAGGCTTCTGC-3′ | 60 |
| Leu²⁹³/Leu⁵¹⁴ | 1 | 5′-GAAGGAAGAGGACTTGCGAAAAAGCTGCAGATT-3′ | 61 |
| | 2 | 5′-AATCTGCAGCTTTTTCGCAAGTCCTCTTCCTTC-3′ | 62 |
| Lys²⁹⁴/Leu⁵¹⁴ | 1 | 5′-GAAGGAAGAGGACTTTTAGCAAAGCTGCAGATT-3′ | 63 |
| | 2 | 5′-AATCTGCAGCTTTGCTAAAAGTCCTCTTCCTTC-3′ | 64 |
| Arg⁴⁹¹/Leu⁵¹⁴ | 1 | 5′-GATATAAATGAAGCGCCTGCATTAGATAATGAG-3′ | 65 |
| | 2 | 5′-CTCATTATCTAATGCAGGCGCTTCATTTATATC-3′ | 66 |
| Leu⁵¹⁴/Asn⁵¹⁶ | 1 | 5′-GATACTCGAGCAGGATATTTAGAAGCGGGAAAGCTT-3′ | 67 |
| | 2 | 5′-AAGCTTTCCCGCTTCTAAATATCCTGCTCGAGTATC-3′ | 68 |

Mutations were confirmed by DNA sequencing of the region containing the mutation. In addition, the genes encoding all LF that demonstrated reduced toxicity were sequenced in their entirety to confirm that only the desired mutations were present.

### Protein Expression and Purification

Mutagenized proteins, they were first transformed into the E. coli dcm^{-/}dam⁻ strain SCS110 to obtain unmethylated plasmid DNA which was then transformed into a non-toxigenic, sporulation-defective strain of B. anthracis, BH445 (Park et al., supra, as described by Quinn et al., supra).

To prepare crude preparations of secreted protein, a single colony of transformed cells was used to inoculate 5 ml FA medium (Singh et al., supra). Cultures were allowed to grow at 37° C. for 14-16 h. Culture supernatant (2 ml) was then concentrated using a centrifugal filter (Microcon 100K MWCO; Millipore) and protein was recovered in 40 μl buffer (20 mM Hepes, pH 7.5, 25 mM NaCl). The concentration of each protein was estimated by direct comparison to Coomassie Blue-stained BSA standards (0.5 and 2.0 mg/ml) after separation on 10-20% SDS-PAGE gels.

To make high-purity preparations of LF and PA, 50 ml cultures were used to inoculate 5 L of FA medium in a BioFlo 100 fermentor (New Brunswick Laboratories) at 37° C., pH 7.4, while sparging with air at 3 L/min and with agitation set to increase from 100 rpm to 400 rpm as level of dO₂ dropped below 50%. After 17-18 h of growth, the cells were removed by centrifugation (3500 g for 30 min., 4° C.), and the supernatant was sterile-filtered and concentrated by tangential flow filtration using a Millipore prep/scale-TFF cartridge with 1 ft² of 30-KDa MWCO polyethersulfone membrane, collecting the filtrate at approximately 50 ml/min under a 1 bar back-pressure.

Expressed protein was purified by ammonium sulfate fractionation and fast pressure liquid chromatography (FPLC) using phenyl sepharose and Q sepharose columns following the procedures of Park et al., supra. The concentration of each protein was estimated using the bicinchoninic acid method (Smith et al., supra) and by densitometric analyses of Coomassie Blue-stained polyacrylamide gels.

Recombinant human MEK1 protein was expressed in Spodoptera frugiperda (Sf9) cells that had been infected with baculovirus containing human MEK1 ligated into the pVL1393 vector backbone (pKM636). Protein was isolated from supernatants of lysed cells and was eluted over 10 column volumes in a linear gradient from 0-500 mM NaCl from a 20 ml Q-Sepharose column. The peak fractions containing MEK proteins were pooled and loaded directly onto a 10 ml Ni-NTA column. After washing the column with 30 mM imidazole, MEK was eluted with 100 mM imidazole. At this point, the eluate was adjusted to 3 μM EDTA, 3 mM MnCl₂, and 2 mM dithiothreitol (DTT), and 25 units of protein phosphatase 1 (New England Biolabs, Beverly, Mass.) were added to the reaction which was allowed to incubate for 4 hrs at 30° C. Samples were then concentrated and applied to a 320 ml Sephacryl 200 column in a buffer of 25 mM HEPES (pH 8.0), 100 mM NaCl, 2 mM DTT and 10% glycerol.

ERK2 protein was expressed in E. coli and purified by FPLC as described earlier (Duesbery et al., supra; Chopra et al., supra). Active B-Raf (Δ1-415) was purchased from Upstate Biotechnology, Inc.

### Cytotoxicity Assays

Cells were grown in 96-well microplates to 70% confluence. To induce lysis, cells were treated with culture medium containing LeTx [PA (0.1 μg/ml) plus LF (0.01-10,000 ng/ml)] and incubated for 3 h at 37° C. At the end of the experiment, cell viability was determined using the CellTiter 96® Aqueous Non-Radioactive Cell Proliferation Assay (Promega, Madison, Wis.) according to the manufacturer's instructions. The concentration of LF required to cause a 50% maximal decrease in absorbance at 570 nm (the EC₅₀) was determined by linear regression.

### PA-Binding and Translocation Assays.

PA-binding and translocation assays were performed as described by Lacy et al. (supra) and quantitated using a Packard Tri-Carb 3100TR liquid scintillation counter.

### MEK Proteolysis and B-Raf Kinase Assays.

To assay MEK cleavage in cells we made lysates of J774A.1 macrophages which had been incubated for 2 h with 0.1 μg/ml PA and 0.01 μg/ml LF or LF mutants. Lysates were separated by denaturing SDS-PAGE and immunoblotted with antibodies raised against the NH₂- or COOH-termini of MEK2 (N-20 and C-16, 1:1000; Santa Cruz Biotechnology, Santa Cruz, Calif.). In vitro MEK cleavage assays were performed using immunoblotting with antibodies raised against MEK (anti-MEK1/2, 1:1000; Cell Signaling) as described earlier (Chopra et al., supra). Alternatively, MEK-cleavage was assayed indirectly by reacting a constant concentration of MEK with varying the amounts of LF, using MEK activity (i.e. ERK phosphorylation) as a readout for LF activity. Briefly, 0.35 μg MEK1 was added to 3 μl cleavage buffer (20 mM 3-(N-morpholino) propanesulfonic acid (pH 7.2), 25 mM β-glycerophosphate, 5 mM ethylene glycol-bis(2-aminoethylether)-N,N,N′,N′-tetraacetic acid, 1 mM sodium orthovanadate, and 1 mM dithiothreitol) in the presence of varying amounts of LF or mutant LF (0.002 μg to 10 μg) and in a total volume of 10 μl. These cleavage reactions were incubated at 30° C. for 10 min. After cooling on ice for 2 min, 10 μl kinase buffer (0.5 mM ATP diluted 9:1 with [γ³²P]ATP (Amersham; 10 mCi/ml, 3000 mCi/mmol)), 75 mM MgCl₂, and 0.4 μg of ERK2] was added and samples were incubated for 10 min at 30° C. After cooling on ice for 2 min one volume of 2× SDS-buffer was added and samples were incubated in a boiling water bath for 3 min. Proteins were then separated by SDS polyacrylamide electrophoresis on 10% gels and ERK2 phosphorylation was quantitated using a Fuji FLA-5000 PhosphorImager.

B-Raf kinase assays were performed as described previously (Copra et al., supra) and quantitated using a Fuji FLA-5000 PhosphorImager. Results were normalized to phosphorylation in the absence of LF and compared using an unpaired Students' t-Test.

### EXAMPLE II

### Site-Directed Mutagenesis of Clustered Aliphatic Residues

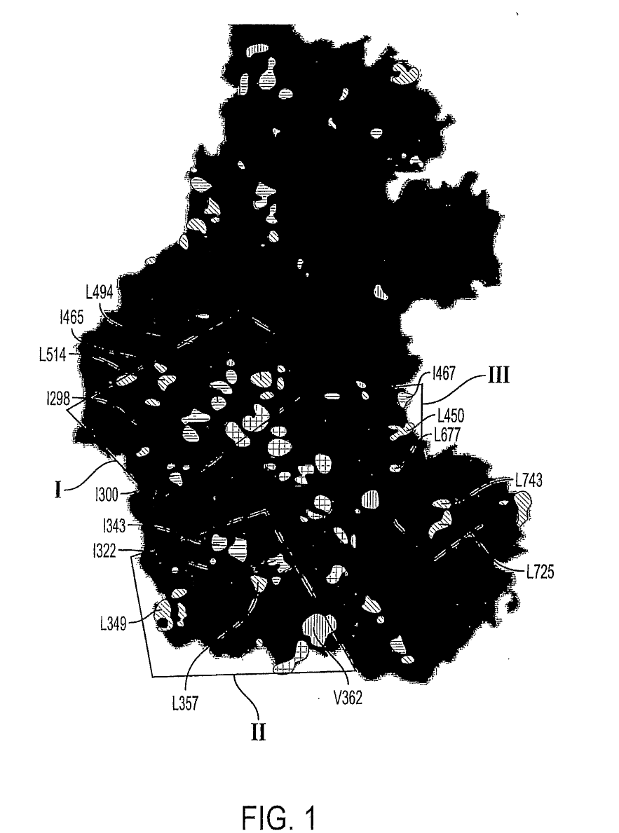

Since a number of the conserved residues in the LFIR are long-chain aliphatic residues, the present inventors conceived that a complementary region on LF would contain clustered aliphatic residues and would lie close to the groove into which the NH₂-terminus of MEK fits. A surface plot of LF shows three distinct clusters of aliphatic residues meeting this requirement (FIG. 1). The first is composed of aliphatic residues (I298, I300, I485, L494, and L514) present in domain II and lies at one end of the catalytic groove. The second (residues I322, I343, L349, L357, and V362) is composed of elements of the second, third, and fourth imperfect repeats in domain III and lies at the opposite end of the catalytic groove. A third cluster present in domain IV (L450, I467, L677, L725, and L743) lies adjacent to the catalytic groove which receives the NH₂-terminus of MEK.

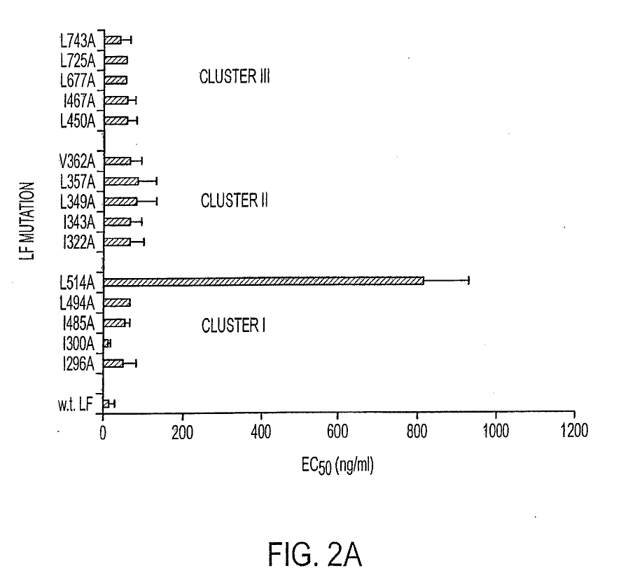

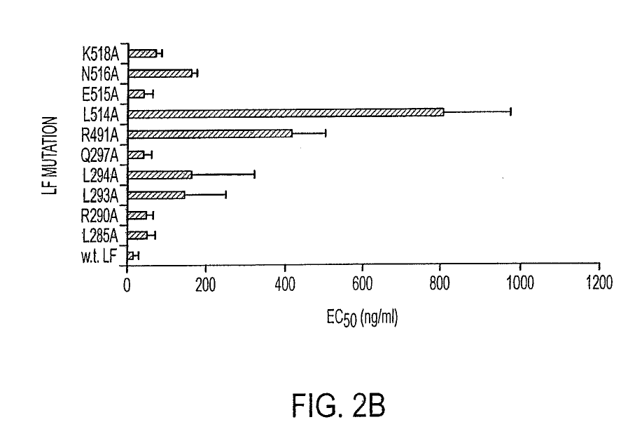

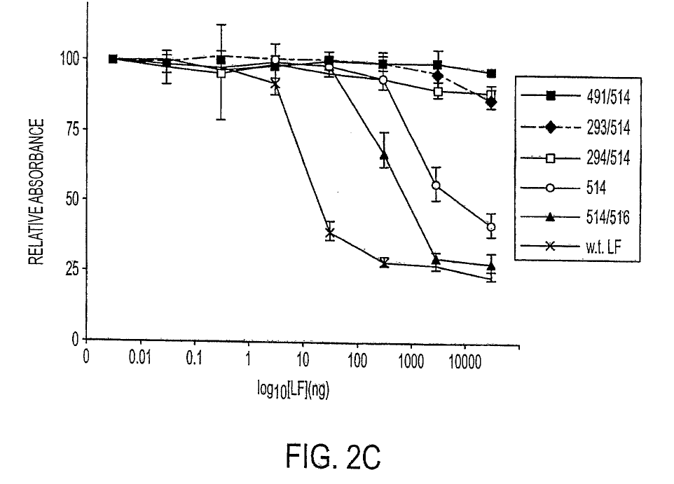

To test this, site-directed mutagenesis was employed to substitute alanine for each of these residues and then evaluated the effects of these mutations upon LF activity using a macrophage toxicity assay (Friedlander, supra). The average concentration of crude preparations of wild-type LF required to cause a 50% maximal decrease in absorbance/cell viability (the EC₅₀) was 15.6±16.7 nM. Mutation of most of the aliphatic residues tested caused a less than a 5-fold reduction in toxicity (12.9 nM≦EC₅₀≦84.3 nM; FIG. 2a) and were judged to have a neutral or marginal role in toxicity. By contrast, alanine substitution of L514 caused a greater than 50-fold reduction in toxicity (EC₅₀=816±137 nM; FIG. 2a).

To determine whether other residues in this region of LF played a role in LF-toxicity, alanine substitutions were made at surface-exposed residues which were in proximity to L514. Of these, L285, R290, Q297, E515, and K518 were judged to have a neutral or marginal role in toxicity (40 nM≦EC₅₀≦65 nM; FIG. 2b). By contrast, alanine substituted at L293, K294, R491, or N516 caused a greater-than-tenfold reduction in toxicity (141 nM EC₅₀≦419 nM; FIG. 2b). Interestingly, while pairwise mutation of L514 and either L293, K294, or R491 completely abrogated LF toxicity (FIG. 2c), pairwise mutation of L514 and N516 instead resulted in toxicity comparable to N516A alone (EC₅₀=200±98 nM for L514A/N516A versus 164±13 nM for N516A; FIG. 2c). These results indicate that subtle perturbations of the surface composition of domain II caused by alanine substitution of these residues can have a substantial impact upon LF toxicity.

### EXAMPLE III

### Point Mutations in Domain II Do Not Reduce LF's Affinity for PA or its Ability to Translocate Across the Plasma Membrane

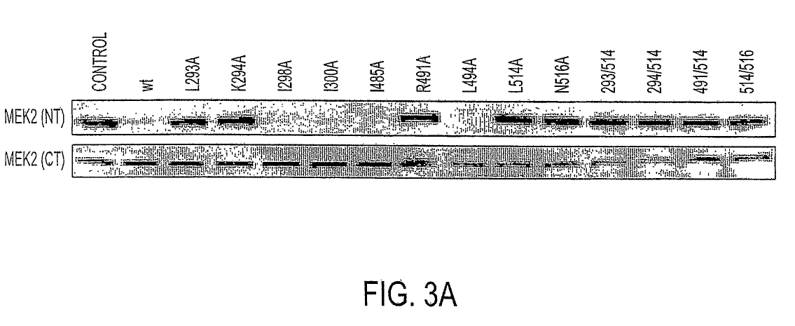

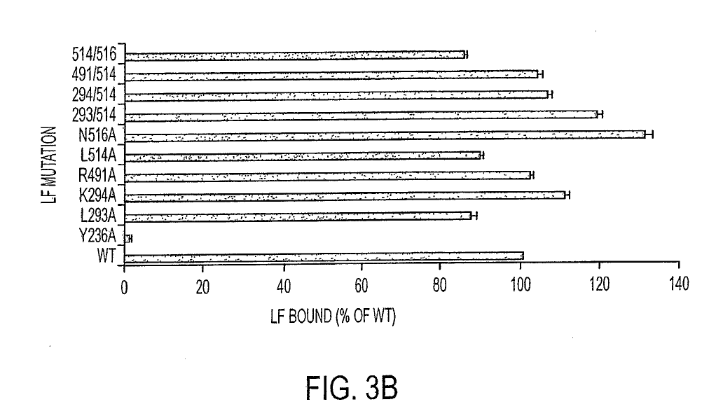

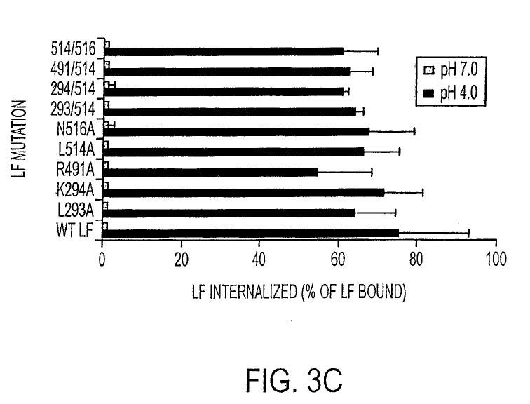

LF is a Zn²⁺-metalloprotease which specifically cleaves the NH₂-termini of mitogen-activated protein kinase kinases. To determine whether clustered residues in domain II are required for LF proteolytic activity we assayed MEK2 cleavage by immunoblotting in J774A.1 macrophages which had been treated for 2 h with PA (0.1 μg/ml) plus wild-type LF or LF containing alanine mutations (0.01 μg/ml) in this region. Of the proteins tested, only wild-type LF and LF containing alanine mutations which had a neutral or marginal effect on toxicity were able to cleave the NH₂-terminus of MEK2 (FIG. 3a). By contrast, L293A, K294A, R491A, L514A, and N516A as well as the double mutants L293A/L514A, K294A/L514A, R491A/L514A, and L514A/N516A caused no or reduced MEK2 cleavage. These results are consistent with our observation that only these residues of domain II play a key role in LF toxicity. However, the preceding assay is cell-based and does not distinguish between decreased toxicity caused by a reduced ability of LF to bind PA, to translocate across the endosomal membrane, or to cleave MEKs. Subsequent analyses were performed to elucidate the mechanism by which these mutations interfere with toxicity.

To test whether our mutant LF were able to bind PA and translocate across the membrane binding and translocation assays were done using [³⁵S]-Met-labeled LF. In these assays LF and PA₆₃ were allowed to bind ANTXR on CHO-K1 cells at 4° C., at which temperature endocytosis does not occur. LF containing an alanine substitution at LF (Y236A) was the negative control—it has was previously shown to be incapable of binding to PA.

After unbound protein was washed away, the cells were treated with low or neutral pH buffer. The low pH buffer mimics the endosomal environment and triggers PA₆₃ pore formation and the subsequent translocation of LF to the cytosol. After this, cells were exposed to pronase to remove any surface-bound label, washed, lysed, and assayed for ³⁵S content. As shown in FIGS. 3b and 3c, wild-type and mutant LF were equally capable of binding PA₆₃ and translocating across the plasma membrane. Consistent with published reports (Lacy et al., supra), LF (Y236A) did not appreciably bind PA₆₃ in the same assays. The ability of wild-type and mutant LF to bind PA was confirmed independently by non-denaturing gel-shift assays using LF and trypsin-nicked PA (PA₆₃) (not shown, but see FIG. 5). These results indicate that loss of toxicity in mutant LF can neither be explained by loss of the ability to bind PA nor by an inability to translocate across a cell membrane.

### EXAMPLE IV

### Point Mutations in Domain II do not Alter LF Proteolytic Activity

Since the preceding assays were performed with relatively crude preparations of protein, it remained a possibility that the results we observed were caused by the effects of contaminants upon mutant LF and not wild-type LF activity. To test this we purified wild-type LF and selected LF double mutants by FPLC and re-assessed the toxicity of these preparations using macrophage cytotoxicity assays. The EC₅₀ of wild-type LF was 10.9±5.9 nM (FIG. 4a) while FPLC-purified L514A proved to be less toxic (EC₅₀>1,000 nM). As noted for crude preparations of protein, the substitution of a second alanine residue for N516 in L514A partially restored the toxicity of this mutant (EC₅₀=427±74 nM). FPLC-purified K294A/L514A and R491/L514A were non-toxic (EC₅₀>>10,000 nM). Since FPLC-purified LF and LF-mutants possess toxicities that are similar to those of crude preparations of the same proteins, it is unlikely that their reduced toxicities may be attributed to the presence of contaminants.

To this point, the analyses indicated that point mutations at clustered residues of domain II reduce the proteolytic activity of LF. To directly test this, the proteolytic activity of FPLC-purified wild-type and mutant LF was tested in vitro by immunoblotting. The control was an FPLC-purified preparation of LF harboring a point mutation in the Zn²⁺-binding domain (E687C), which has been previously characterized as being non-toxic Klimpel, K R et al. (1994) Mol Microbiol 13:1093-1100 and proteolytically inactive (Duesbery et al., 1998, supra)). Incubation of 0.2 μg wild-type LF, but not E687C, with 0.2 μg NH₂-terminally His₆-tagged MEK1 increased the electrophoretic mobility of MEK1, consistent with NH₂-terminal proteolysis as described. Unexpectedly, none of the mutant LF showed reduced proteolytic activity towards MEK (FIG. 4b). However, this sort of cleavage assay is qualitative in nature and may not reveal partial reduction of proteolytic activity.

Modified cleavage assays were performed in the concentration of LF was varied in the presence of a fixed amount of MEK and the kinase activity of MEK towards ERK was used as an indirect, but quantifiable, measure of proteolysis. Wild-type LF caused a robust inhibition of MEK activity and resulted in a 50% suppression of ERK phosphorylation at a molar ratio (LF:MEK) of 0.5±0.3 (FIG. 4b). By contrast, the control E687C had no effect on MEK activity (except when present in excess). Consistent with the observed toxicity of these mutants, K294A/L514A and R491A/L514A showed markedly reduced proteolytic activity, showing 50% suppression of ERK phosphorylation at molar ratios of 1.9±1.1 and 1.6±0.4, respectively. Unexpectedly, L514A and L514A/N516A possessed proteolytic activity which was comparable to wild-type activity, causing a 50% suppression of ERK phosphorylation at a molar ratios of 0.9±0.1 and 0.8±0.3, respectively. Thus, while clustered point mutations in domain II decrease LF toxicity, this loss may not be entirely attributed to decreased proteolytic activity.

An alternative explanation for the foregoing observations is that decreased LF toxicity may be caused by a loss of substrate affinity that is independent of proteolytic activity. In lieu of a direct assay of LF binding to MEK, the present inventor and colleagues previously demonstrated that LF could competitively inhibit B-Raf phosphorylation of MEK and that this inhibition was independent of its proteolytic activity. The interpretation of these results was that LF and B-Raf bound to adjacent or overlapping epitopes on MEK. To determine whether point mutations at clustered residues of domain II reduced the affinity of LF for MEK. in vitro B-Raf-mediated MEK phosphorylation was assayed in the presence of LF or LF mutants. As reported earlier, LF caused an approximately 35% inhibition of MEK phosphorylation by B-Raf and this effect was independent of LF proteolytic activity since E687C also inhibited MEK phosphorylation by B-Raf (FIG. 4c). Interestingly, whereas L514A and L514A/N516A had an effect that was similar to that of wild type LF on MEK phosphorylation, K294A/L514A and R491A/L514A showed significantly reduced inhibition, blocking only 10±7% (p=0.014, df=6) and 2±5% (p=0.0026, df=6) of Raf-mediated phosphorylation, respectively (FIG. 4c). These results indicated that decreased LF toxicity resulting from point mutations in clustered residues in domain II may be attributed in part to decreased ability interact with MEK.

### Discussion of Examples I-IV

LF is the principal virulence factor of anthrax toxin (Cataldi, A et al. (1990) Mol. Microbiol. 4:1111-17; Pezard, C et al. (1991) Infec Immun 59:3472-77; Pezard, C et al. (1993) J. Gen. Microbiol. 139:2459-63). To date, its only identified substrates are members of the MEK family of protein kinases. Consequently, the interaction between LF and MEK is an important concern for understanding the pathogenesis of anthrax as well as in the design of targeted therapeutic agents. This studies described above were undertaken to identify regions of LF which are required for interaction with MEK. As a starting point, the present inventor reasoned that since a number of the conserved residues in the LFIR are long-chain aliphatic residues, any region of LF with which it associated would (i) contain a cluster of surface-exposed aliphatic residues and (ii) lie adjacent to the catalytic groove where the active site complex would form. Regardless of the physiological relevance of these assumptions, tests of this hypothesis led to the identification of a single residue (L514) in domain II which, when replaced by an alanine residue, resulted in a substantial reduction in LF toxicity. Further alanine-substitution in the vicinity of L514 identified four additional residues which also play a role in LF toxicity. Though separated in primary sequence, the tertiary structure of LF brings these five residues side-by-side in a focused region which lies at one end of the groove which forms between domains III and IV and contains the active site (see FIG. 6).

What role does this region play in LF toxicity? One key observation is that although mutant LF (i.e. L514A and L514A/N516A) were incapable of cleaving MEK in cell-based assays, they did so in vitro. This indicates that these mutants are sensitive to the context in which they encounter their substrate MEKs. In cells, the spatial distribution and accessibility of MEKs are influenced by scaffolding proteins such as MP1 (Schaeffer, H J et al. (1998) Science 281:1668-1671) and JIP-1 (Whitmarsh, A J et al. (1998) Science 281:1671-74). In addition, cellular MEKs may be modified post-translationally (e.g. by phosphorylation) and can associate with their cognate MAPKs as well as other regulatory molecules such as B-Raf. Any of these factors may limit the ability of mutant LF to bind and cleave MEKs in cells. Indeed, while the MEK1 scaffolding protein MP1 can associate with both recombinant MEK1 and MEK2 in vitro, it can only bind MEK1 in cells (Schaeffer et al., supra). While the present invention is not intended to be bound by potential mechanism(s), the simplest interpretation of the present observations is that the region herein identified defines a site which is necessary for LF to associate into a productive complex with MEKs. Several observations support this conception: (i) the effect of the mutations was specific; only mutations in this region, but not in clusters II or III, decreased LF toxicity, (ii) decreased toxicity was accompanied by decreased proteolysis of MEK2 in cells, (iii) mutations in this region did not alter “other” functions: binding to PA or translocation of LF across the cell membrane, (iv) the LF mutants L514A and L514A/N516A possessed in vitro proteolytic activity which was comparable to that of wild-type LF, (v) the LF mutants K294A/L514A and R491A/L514A display reduced ability to competitively inhibit B-Raf phosphorylation of MEK, and (vi) the region identified on domain II is spatially distinct from the active site and thus is not likely to directly participate in substrate proteolysis. Because mutant LF not only retained the ability to bind PA and internalize into cells but also (in the case of L514A and L514A/N516A) possessed wild-type levels of proteolytic activity, an explanation that that the mutations introduce gross, structural changes in LF with nonspecific effects.

Understanding the precise role this region plays in promoting the association of LF into a productive complex with MEKs requires further research. This region may be required to direct LF to MEKs within cells. In this case, mutations in this region of domain II would reduce the ability of LF to associate with proteins which co-localize with MEKs. Alternatively, this region may play a direct role in binding MEKs. The latter possibility is supported by the observations that the LF mutants K294A/L514A and R491A/L514A display reduced ability to competitively inhibit B-Raf phosphorylation of MEK. Moreover, indirect evidence supports the conclusion view that LF and MEK interact at sites outside the active site. Using yeast two-hybrid analysis to identify binding partners of LF, Vitale et al. (supra) isolated cDNA encoding MEK2 which lacked the NH₂-terminal cleavage site. In addition, the present inventor and colleagues earlier demonstrated the existence of a conserved region located in C-terminus of MEK1 which is required for LF-mediated proteolysis of MEKs (Chopra, A P et al., 2003, supra).

Recent publications have identified lead compounds which may be adapted for use as small molecule inhibitors of LF activity (Dell'Aica, I et al. (2004) EMBO Rep 5:418-22; Min, D H et al. (2004) Nat Biotechnol 22:717-23; Turk, B E et al. (2004) Nat Struct Mol Biol 11:60-6; Tonello, F et al. (2002) Nature 418:386). These molecules were initially identified by LF cleavage (proteolysis) assays that used optimized peptide substrates which mimic the NH₂-terminal cleavage site on MEKs. However, as shown herein, sites outside the active site complex on both LF and MEK are required for efficient proteolysis of MEK.

Thus, according to the present invention, novel and more effective anthrax therapeutics are molecules that are targeted to the region of LF defined by these residues, and which are used either alone or in combination with those identified molecules which target LF's active site.

The references cited above are all incorporated by reference herein, whether specifically incorporated or not.

Having now fully described this invention, it will be appreciated by those skilled in the art that the same can be performed within a wide range of equivalent parameters, concentrations, and conditions without departing from the spirit and scope of the invention and without undue experimentation.

1. A mutant or variant anthrax lethal factor (LF) polypeptide in which between one and five amino acid residues in domain II that is important for interaction with the LF substrate MEK-I or MEK-2, are either substituted, deleted, or chemically derivatized such that the polypeptide is inhibited compared to normal LF in binding to and interacting with said MEK, the residues selected from the group consisting of L293, K294, R491, L514 and N516.

2. The mutant or variant LF of claim 1 in which at least two amino acid residues in domain II is substituted or mutated, which two residues are selected from the group consisting of L514/L293, L514/K294 and L514/R491.

3. The mutant or variant LF of claim 1 wherein said one to five amino acid residues are substituted with Ala or GIy.

4. The mutant or variant LF of claim 2 wherein said at least two amino acid residues is substituted with Ala or GIy.

5. The mutant or variant LF of claim 3 which is selected from the group consisting of L293A, K294A, R491A, L514A, N516A, L514A/L293A, L514A/K294A and L514A/R491A.

6. A fragment of the mutant or variant of claim 1 corresponding to domain IIa or domain IIb of said LF, or a mixture thereof.

7. The fragment or mixture of claim 6 wherein said domain IIa or domain IIb consists essentially of SEQ ID NO:4 or SEQ ID NO:6.

8. An isolated nucleic acid molecule that encodes the mutant or variant LF polypeptide of claim 1.

9. An isolated nucleic acid molecule that encodes the fragment of claim 7 or 8.

10. A method for screening a test sample comprising an agent or compound being tested for its ability to inhibit the binding interaction of LF and MEK independent of any effect on LF-mediated proteolysis of MEK, comprising:
(a) contacting a test sample with LF and a MEK protein; and
(b) assaying for the binding of LF to MEK;
(c) comparing said binding to the binding of LF in the absence of said test sample, wherein, if the binding measured in (a) is lower than the binding measure in (b), said agent or compound is an inhibitor of LF-MEK binding.

11. The method claim 11 further comprising the step of comparing the binding in step (b) with the binding to MEK of an LF mutant, variant or fragment according to claim 1.

12. The method of claim 10 further comprising testing the ability of the sample to inhibit MEK proteolysis, wherein if the compound is positive in inhibiting said binding and negative in inhibiting said proteolysis, it is a binding inhibitor.

13. A method for screening a sample or multiplicity of samples comprising an agent or compound being tested for (i) its ability to inhibit the binding interaction of LF and MEK and (ii) its ability to inhibit LF-mediated proteolysis of MEK and, comprising:
(a) contacting a test sample with LF and a MEK protein,
(b) assaying for the binding of LF to MEK; and
(c) comparing said binding to the binding of LF in the absence of said test sample,
(d) independently of the assay of step (b), assaying for the proteolysis of MEK by LF in the presence of said test sample or samples, and
(e) comparing said proteolysis in (d) to the proteolysis of MEK by LF in the absence of said test sample, wherein, if the binding measured in (a) is lower than the binding measure in (b), and the proteolysis measured in (d) is lower that the proteolysis measure in (e) said agent or compound in said sample or said agents or compounds in said multiplicity of samples are inhibitors of LF-MEK binding and LF-mediated MEK proteolysis.

14. The method of claim 13 further comprising comparing the binding in step (b) with the binding to MEK of an LF mutant, variant or fragment according to claim 1.

15. An immunogenic or vaccine composition comprising:
(a) the mutant or variant LF of claim 1; and
(b) an immunologically acceptable carrier or excipient.

16. An immunogenic or vaccine composition comprising:
(a) the nucleic acid molecule of claim 9, and
(b) an immunologically acceptable carrier or excipient.

17. A method of inducing LF specific immunity in a subject comprising administering to the subject an immunogenically effective amount of the composition of claim 15.

18. A method of inducing LF specific immunity in a subject comprising administering to the subject an immunogenically effective amount of the composition of claim 16.


SEQUENCE LISTING (68 sequences)

SEQ ID NO: 1 - DNA, 2430 bp, Bacillus anthracis
  CDS (1)..(2427)
atgaatataaaaaaagaatttataaaagtaattagtatgtcatgtttagtaacagcaattactttgagtggtcccgtctttatcccccttgtacagggggcgggcggtcatggtgatgtaggtatgcacgtaaaagagaaagagaaaaataaagatgagaataagagaaaagatgaagaacgaaataaaacacaggaagagcatttaaaggaaatcatgaaacacattgtaaaaatagaagtaaaaggggaggaagctgttaaaaaagaggcagcagaaaagctacttgagaaagtaccatctgatgttttagagatgtataaagcaattggaggaaagatatatattgtggatggtgatattacaaaacatatatctttagaagcattatctgaagataagaaaaaaataaaagacatttatgggaaagatgctttattacatgaacattatgtatatgcaaaagaaggatatgaacccgtacttgtaatccaatcttcggaagattatgtagaaaatactgaaaaggcactgaacgtttattatgaaataggtaagatattatcaagggatattttaagtaaaattaatcaaccatatcagaaatttttagatgtattaaataccattaaaaatgcatctgattcagatggacaagatcttttatttactaatcagcttaaggaacatcccacagacttttctgtagaattcttggaacaaaatagcaatgaggtacaagaagtatttgcgaaagcttttgcatattatatcgagccacagcatcgtgatgttttacagctttatgcaccggaagcttttaattacatggataaatttaacgaacaagaaataaatctatccttggaagaacttaaagatcaacggatgctgtcaagatatgaaaaatgggaaaagataaaacagcactatcaacactggagcgattctttatctgaagaaggaagaggacttttaaaaaagctgcagattcctattgagccaaagaaagatgacataattcattctttatctcaagaagaaaaagagcttctaaaaagaatacaaattgatagtagtgattttttatctactgaggaaaaagagtttttaaaaaagctacaaattgatattcgtgattctttatctgaagaagaaaaagagcttttaaatagaatacaggtggatagtagtaatcctttatctgaaaaagaaaaagagtttttaaaaaagctgaaacttgatattcaaccatatgatattaatcaaaggttgcaagatacaggagggttaattgatagtccgtcaattaatcttgatgtaagaaagcagtataaaagggatattcaaaatattgatgctttattacatcaatccattggaagtaccttgtacaataaaatttatttgtatgaaaatatgaatatcaataaccttacagcaaccctaggtgcggatttagttgattccactgataatactaaaattaatagaggtattttcaatgaattcaaaaaaaatttcaaatatagtatttctagtaactatatgattgttgatataaatgaaaggcctgcattagataatgagcgtttgaaatggagaatccaattatcaccagatactcgagcaggatatttagaaaatggaaagcttatattacaaagaaacatcggtctggaaataaaggatgtacaaataattaagcaatccgaaaaagaatatataaggattgatgcgaaagtagtgccaaagagtaaaatagatacaaaaattcaagaagcacagttaaatataaatcaggaatggaataaagcattagggttaccaaaatatacaaagcttattacattcaacgtgcataatagatatgcatccaatattgtagaaagtgcttatttaatattgaatgaatggaaaaataatattcaaagtgatcttataaaaaaggtaacaaattacttagttgatggtaatggaagatttgtttttaccgatattactctccctaatatagctgaacaatatacacatcaagatgagatatatgagcaagttcattcaaaagggttatatgttccagaatcccgttctatattactccatggaccttcaaaaggtgtagaattaaggaatgatagtgagggttttatacacgaatttggacatgctgtggatgattatgctggatatctattagataagaaccaatctgatttagttacaaattctaaaaaattcattgatatttttaaggaagaagggagtaatttaacttcgtatgggagaacaaatgaagcggaattttttgcagaagcctttaggttaatgcattctacggaccatgctgaacgtttaaaagttcaaaaaaatgctccgaaaactttccaatttattaacgatcagattaagttcattattaactcataa

SEQ ID NO: 2 - protein, 809 aa, Bacillus anthracis
MNIKKEFIKVISMSCLVTAITLSGPVFIPLVQGAGGHGDVGMHVKEKEKNKDENKRKDEERNKTQEEHLKEIMKHIVKIEVKGEEAVKKEAAEKLLEKVPSDVLEMYKAIGGKIYIVDGDITKHISLEALSEDKKKIKDIYGKDALLHEHYVYAKEGYEPVLVIQSSEDYVENTEKALNVYYEIGKILSRDILSKINQPYQKFLDVLNTIKNASDSDGQDLLFTNQLKEHPTDFSVEFLEQNSNEVQEVFAKAFAYYIEPQHRDVLQLYAPEAFNYMDKFNEQEINLSLEELKDQRMLSRYEKWEKIKQHYQHWSDSLSEEGRGLLKKLQIPIEPKKDDIIHSLSQEEKELLKRIQIDSSDFLSTEEKEFLKKLQIDIRDSLSEEEKELLNRIQVDSSNPLSEKEKEFLKKLKLDIQPYDINQRLQDTGGLIDSPSINLDVRKQYKRDIQNIDALLHQSIGSTLYNKIYLYENMNINNLTATLGADLVDSTDNTKINRGIFNEFKKNFKYSISSNYMIVDINERPALDNERLKWRIQLSPDTRAGYLENGKLILQRNIGLEIKDVQIIKQSEKEYIRIDAKVVPKSKIDTKIQEAQLNINQEWNKALGLPKYTKLITFNVHNRYASNIVESAYLILNEWKNNIQSDLIKKVTNYLVDGNGRFVFTDITLPNIAEQYTHQDEIYEQVHSKGLYVPESRSILLHGPSKGVELRNDSEGFIHEFGHAVDDYAGYLLDKNQSDLVTNSKKFIDIFKEEGSNLTSYGRTNEAEFFAEAFRLMHSTDHAERLKVQKNAPKTFQFINDQIKFIINS

SEQ ID NO: 3 - DNA, 240 bp, Bacillus anthracis
  CDS (1)..(240)
cggatgctgtcaagatatgaaaaatgggaaaagataaaacagcactatcaacactggagcgattctttatctgaagaaggaagaggacttttaaaaaagctgcagattcctattgagccaaagaaagatgacataattcattctttatctcaagaagaaaaagagcttctaaaaagaatacaaattgatagtagtgattttttatctactgaggaaaaagagtttttaaaaaagctacaa

SEQ ID NO: 4 - protein, 80 aa, Bacillus anthracis
RMLSRYEKWEKIKQHYQHWSDSLSEEGRGLLKKLQIPIEPKKDDIIHSLSQEEKELLKRIQIDSSDFLSTEEKEFLKKLQ

SEQ ID NO: 5 - DNA, 492 bp, Bacillus anthracis
  CDS (1)..(492)
gatattaatcaaaggttgcaagatacaggagggttaattgatagtccgtcaattaatcttgatgtaagaaagcagtataaaagggatattcaaaatattgatgctttattacatcaatccattggaagtaccttgtacaataaaatttatttgtatgaaaatatgaatatcaataaccttacagcaaccctaggtgcggatttagttgattccactgataatactaaaattaatagaggtattttcaatgaattcaaaaaaaatttcaaatatagtatttctagtaactatatgattgttgatataaatgaaaggcctgcattagataatgagcgtttgaaatggagaatccaattatcaccagatactcgagcaggatatttagaaaatggaaagcttatattacaaagaaacatcggtctggaaataaaggatgtacaaataattaagcaatccgaaaaagaatatataaggattgatgcgaaagtagtg

SEQ ID NO: 6 - protein, 164 aa, Bacillus anthracis
DINQRLQDTGGLIDSPSINLDVRKQYKRDIQNIDALLHQSIGSTLYNKIYLYENMNINNLTATLGADLVDSTDNTKINRGIFNEFKKNFKYSISSNYMIVDINERPALDNERLKWRIQLSPDTRAGYLENGKLILQRNIGLEIKDVQIIKQSEKEYIRIDAKVV

SEQ ID NO: 7 - DNA, 34 bp, Artificial Sequence
  primer
gtcaagatatgaaaaagcggaaaagataaaacag

SEQ ID NO: 8 - DNA, 34 bp, Artificial Sequence
  primer
ctgttttatcttttccgctttttcatatcttgac

SEQ ID NO: 9 - DNA, 34 bp, Artificial Sequence
  primer
gcactatcaacacgcgagcgattctttatctgaa

SEQ ID NO: 10 - DNA, 34 bp, Artificial Sequence
  primer
ttcagataaagaatcgctcgcgtgttgatagtgc

SEQ ID NO: 11 - DNA, 34 bp, Artificial Sequence
  primer
gtggagcgattctgcgtctgaagaaggaagagga

SEQ ID NO: 12 - DNA, 34 bp, Artificial Sequence
  primer
tcctcttccttcttcagacgcagaatcgctccac

SEQ ID NO: 13 - DNA, 30 bp, Artificial Sequence
  primer
gaagaaggagcgggacttttaaaaaagctg

SEQ ID NO: 14 - DNA, 30 bp, Artificial Sequence
  primer
cttcttcctcgccctgaaaattttttcgac

SEQ ID NO: 15 - DNA, 33 bp, Artificial Sequence
  primer
gaaggaagaggacttgcgaaaaagctgcagatt

SEQ ID NO: 16 - DNA, 33 bp, Artificial Sequence
  primer
aatctgcagctttttcgcaagtcctcttccttc

SEQ ID NO: 17 - DNA, 33 bp, Artificial Sequence
  primer
gaaggaagaggacttttagcaaagctgcagatt

SEQ ID NO: 18 - DNA, 33 bp, Artificial Sequence
  primer
aatctgcagctttgctaaaagtcctcttccttc

SEQ ID NO: 19 - DNA, 33 bp, Artificial Sequence
  primer
ggacttttaaaaaagctggcaattcctattgag

SEQ ID NO: 20 - DNA, 33 bp, Artificial Sequence
  primer
ctcaataggaattgccagcttttttaaaagtcc

SEQ ID NO: 21 - DNA, 39 bp, Artificial Sequence
  primer
ggacttttaaaaaagctgcaggcacctattgagccaaag

SEQ ID NO: 22 - DNA, 39 bp, Artificial Sequence
  primer
ctttggctcaataggtgcctgcagcttttttaaaagtcc

SEQ ID NO: 23 - DNA, 31 bp, Artificial Sequence
  primer
gctgcagattcctgcggagccaaagaaagat

SEQ ID NO: 24 - DNA, 31 bp, Artificial Sequence
  primer
atctttctttggctccgcaggaatctgcagc

SEQ ID NO: 25 - DNA, 36 bp, Artificial Sequence
  primer
gagcttctaaaaagagcacaaattgatagtagtgat

SEQ ID NO: 26 - DNA, 36 bp, Artificial Sequence
  primer
atcactactatcaatttgtgctctttttagaagctc

SEQ ID NO: 27 - DNA, 39 bp, Artificial Sequence
  primer
gagtttttaaaaaagctacaagcagatattcgtgattct

SEQ ID NO: 28 - DNA, 39 bp, Artificial Sequence
  primer
agaatcacgaatatctgcttgtagcttttttaaaaactc

SEQ ID NO: 29 - DNA, 36 bp, Artificial Sequence
  primer
gatattcgtgattctgcatctgaagaagaaaaagag

SEQ ID NO: 30 - DNA, 36 bp, Artificial Sequence
  primer
ctctttttcttcttcagatgcagaatcacgaatatc

SEQ ID NO: 31 - DNA, 33 bp, Artificial Sequence
  primer
gaaaaagagcttgcaaatagaatacaggtggat

SEQ ID NO: 32 - DNA, 33 bp, Artificial Sequence
  primer
atccacctgtattctatttgcaagctctttttc

SEQ ID NO: 33 - DNA, 39 bp, Artificial Sequence
  primer
gagcttttaaatagaatacaggcagatagtagtaatcct

SEQ ID NO: 34 - DNA, 39 bp, Artificial Sequence
  primer
aggattactactatctgcctgtattctatttaaaagctc

SEQ ID NO: 35 - DNA, 37 bp, Artificial Sequence
  primer
gaatatcaataaccttacagcaaccgcaggtgcggat

SEQ ID NO: 36 - DNA, 37 bp, Artificial Sequence
  primer
atccgcacctgcggttgctgtaaggttattgatattc

SEQ ID NO: 37 - DNA, 36 bp, Artificial Sequence
  primer
gataatactaaaattaatagaggtgcattcaatgaa

SEQ ID NO: 38 - DNA, 36 bp, Artificial Sequence
  primer
ttcattgaatgcacctctattaattttagtattatc

SEQ ID NO: 39 - DNA, 37 bp, Artificial Sequence
  primer
gagtatttctagtaactatatggcagttgatataaat

SEQ ID NO: 40 - DNA, 37 bp, Artificial Sequence
  primer
atttatatcaactgccatatagttactagaaatactc

SEQ ID NO: 41 - DNA, 33 bp, Artificial Sequence
  primer
gatataaatgaagcgcctgcattagataatgag

SEQ ID NO: 42 - DNA, 33 bp, Artificial Sequence
  primer
ctcattatctaatgcaggcgcttcatttatatc

SEQ ID NO: 43 - DNA, 36 bp, Artificial Sequence
  primer
gatataaatgaaaggcctgcagcagataatgagcgt

SEQ ID NO: 44 - DNA, 36 bp, Artificial Sequence
  primer
acgctcattatctgctgcaggcctttcatttatatc

SEQ ID NO: 45 - DNA, 36 bp, Artificial Sequence
  primer
gatactcgagcaggagcgttagaaaatggaaagctt

SEQ ID NO: 46 - DNA, 36 bp, Artificial Sequence
  primer
aagctttccattttctaacgctcctgctcgagtatc

SEQ ID NO: 47 - DNA, 36 bp, Artificial Sequence
  primer
gatactcgagcaggatatgcggaaaatggaaagctt

SEQ ID NO: 48 - DNA, 36 bp, Artificial Sequence
  primer
aagctttccattttccgcatatcctgctcgagtatc

SEQ ID NO: 49 - DNA, 36 bp, Artificial Sequence
  primer
gatactcgagcaggatatttagcgaatggaaagctt

SEQ ID NO: 50 - DNA, 36 bp, Artificial Sequence
  primer
aagctttccattcgctaaatatcctgctcgagtatc

SEQ ID NO: 51 - DNA, 36 bp, Artificial Sequence
  primer
gatactcgagcaggatatttagaagcgggaaagctt

SEQ ID NO: 52 - DNA, 36 bp, Artificial Sequence
  primer
aagctttcccgcttctaaatatcctgctcgagtatc

SEQ ID NO: 53 - DNA, 36 bp, Artificial Sequence
  primer
gcaggatatttagaaaatggagcgcttatattacaa

SEQ ID NO: 54 - DNA, 36 bp, Artificial Sequence
  primer
ttgtaatataagcgctccattttctaaatatcctgc

SEQ ID NO: 55 - DNA, 39 bp, Artificial Sequence
  primer
ggaccttcaaaaggtgtagaagcaaggaatgatagtgag

SEQ ID NO: 56 - DNA, 39 bp, Artificial Sequence
  primer
ctcactatcattccttgcttctacaccttttgaaggtcc

SEQ ID NO: 57 - DNA, 36 bp, Artificial Sequence
  primer
gaagggagtaatgcaacttcgtatgggagaacaaat

SEQ ID NO: 58 - DNA, 36 bp, Artificial Sequence
  primer
atttgttctcccatacgaagttgcattactcccttc

SEQ ID NO: 59 - DNA, 36 bp, Artificial Sequence
  primer
gcagaagcctttagggcgatgcattctacggaccat

SEQ ID NO: 60 - DNA, 36 bp, Artificial Sequence
  primer
atggtccgtagaatgcatcgccctaaaggcttctgc

SEQ ID NO: 61 - DNA, 33 bp, Artificial Sequence; identical to SEQ ID NO: 15
  primer

SEQ ID NO: 62 - DNA, 33 bp, Artificial Sequence; identical to SEQ ID NO: 16
  primer

SEQ ID NO: 63 - DNA, 33 bp, Artificial Sequence; identical to SEQ ID NO: 17
  primer

SEQ ID NO: 64 - DNA, 33 bp, Artificial Sequence; identical to SEQ ID NO: 18
  primer

SEQ ID NO: 65 - DNA, 33 bp, Artificial Sequence; identical to SEQ ID NO: 41
  primer

SEQ ID NO: 66 - DNA, 33 bp, Artificial Sequence; identical to SEQ ID NO: 42
  primer

SEQ ID NO: 67 - DNA, 36 bp, Artificial Sequence; identical to SEQ ID NO: 51
  primer

SEQ ID NO: 68 - DNA, 36 bp, Artificial Sequence; identical to SEQ ID NO: 52
  primer